(19) United States
(12) Patent Application Publication
(10) Pub. No.: US 2018/0052105 A1
(43) Pub. Date: Feb. 22, 2018

(54) SULFANE SULFUR-SELECTIVE FLUORESCENT PROBE

(71) Applicant: The University of Tokyo, Tokyo (JP)

(72) Inventors: Kenjiro HANAOKA, Tokyo (JP); Kazuhito SHIMAMOTO, Tokyo (JP); Yasuteru URANO, Tokyo (JP); Yoko TAKANO, Tokyo (JP)

(21) Appl. No.: 15/552,175

(22) PCT Filed: Jan. 21, 2016
(86) PCT No.: PCT/JP2016/051702

§ 371 Date: Aug. 18, 2017

Related U.S. Application Data
(60) Provisional application No. 62/118,558, filed on Feb. 20, 2015.

(51) Int. Cl. G01N 21/64; C09B 11/28; C12Q 1/02; G01N 33/58; G01N 33/84

(52) U.S. Cl. CPC G01N 21/64; C09B 11/28; G01N 21/3151; G01N 33/58; G01N 33/84; C12Q 1/02

(57) ABSTRACT

Fluorescent probes and their salts show sufficient solubility in water and detect sulfane sulfur in vivo. The fluorescent probes can have the following structure:

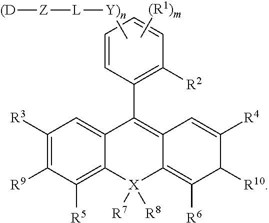

### TECHNICAL FIELD

The present invention relates to a novel fluorescent probe capable of detecting sulfane sulfur selectively.

### BACKGROUND ART

Active sulfur molecules produced in vivo are reported, due to their high reactivity, to modify proteins and signal molecules and to participate in various physiological functions, and have recently drawn attention. Here, the term active sulfur molecule means a molecule having a highly reactive sulfur atom such as hydrogen sulfide (H₂S) and a sulfur atom with a valence of 0 (S⁰, sulfane sulfur).

H₂S has drawn attention and has been studied since the 1990s as an active sulfur molecule. (Non-patent References 1 and 2) Moreover, in addition to H₂S, a sulfur atom having a valence of 0 (S⁰, sulfane sulfur) is drawing attention as a more highly reactive sulfur atom (Non-patent Reference 3). Cystathionine β-synthase (CBS), cystathionine γ-lyase (CSE), 3-mercaptopyruvate sulfur transferase (3MST), and the like are reported as enzymes that produce active sulfur molecules in vivo (see FIG. 1). How important H₂S and sulfane sulfur are in vivo and how much H₂S and sulfane sulfur these enzymes produce in vivo are still matters under discussion. It is therefore very important for the advance of this research field to develop research tools such as a technique for detecting active sulfur molecules in vivo selectively and at good sensitivity.

It became clear in 2014 that cysteine persulfide which includes sulfane sulfur is produced from CSE when cystine is used as the substrate, and sulfane sulfur was shown to actually be present in vivo (Non-patent Reference 4).

Techniques for detecting sulfane sulfur in cells have therefore drawn attention in recent years.

There are three methods for detecting sulfane sulfur: absorption spectrometry, monobromobimane, and fluorescent probe.

Absorption Spectrometry

Absorption spectrometry is a method that quantifies the thiocyanate produced from cyanide and sulfane sulfur by Fe³⁺, and sulfane sulfur is detected by the following scheme. The low sensitivity is a problem in absorption spectrometry.

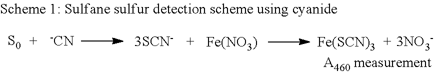

Monobromobimane

Detection by monobromobimane is a method that analyzes the reaction product of monobromobimane, which is an electrophilic fluorescent reagent, and an active sulfur molecule by LC-MS (Scheme 2). Although this method is advantageous in that it can simultaneously quantify which active sulfur molecules are present to what extent at good sensitivity, it is unsuited to real time measurement since invasive procedures such as homogenization are required, as is the case with absorption spectrometry.

Scheme 2: Sulfane Sulfur Detection Scheme Using Monobromobimane

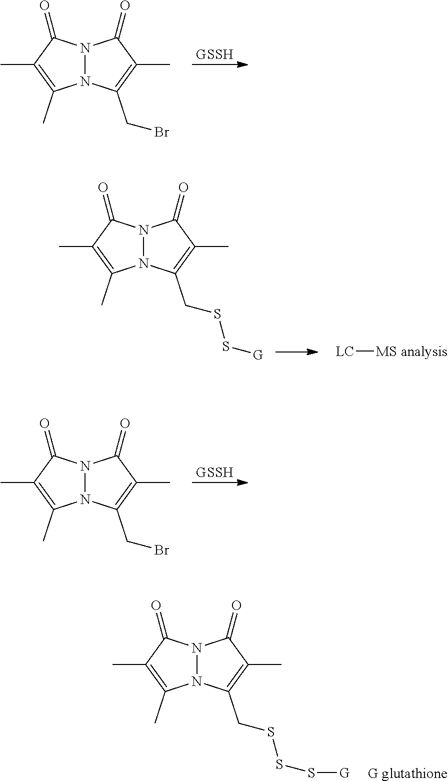

Fluorescent Probe

Fluorescent imaging using a fluorescent probe is superior in that it permits measurement at high spatiotemporal resolution, easily and at good sensitivity, while cells remain in a living state, and is a widely used method. The following two types of sulfane sulfur-selective fluorescent probes have been reported to date, both by Ming et al. (see FIG. 2). (Non-patent References 5 and 6)

(1) SSP

Persulfide is produced by the electrophilic reaction of sulfane sulfur, and a fluorophore is released by nucleophilic attack on a nearby ester structure.

(2) DSP

This probe utilizes the nucleophilic reaction of hydrogen persulfide (H—S—Sₙ—S—H). Persulfide is produced by a nucleophilic substitution reaction, and a fluorophore is released by nucleophilic attack on a nearby ester structure. DSP is a fluorescent probe that does not respond to molecules in which sulfur atoms at the ends are modified (R—S—Sₙ—S—R, R—S—Sₙ—H) and responds selectively to hydrogen persulfide of which sulfur atoms at the ends are not modified.

### PRIOR ART REFERENCES

### Non-Patent References

Non-patent Reference 1: Neurosci., 1996, 16, 1066-1071
Non-patent Reference 2: Biochem. Biophysics. Res. Communed., 1997, 237, 527-531
Non-patent Reference 3: Anal. Biochem., 2011, 413, 1-7
Non-patent Reference 4: Proc. Natl. Acad. Sci. USA, 2014, 111, 7606-7611
Non-patent Reference 5: Chem. Sci., 2013, 4, 2892-2896
Non-patent Reference 6: J. Am. Chem. Soc., 2014, 136, 7257-7260

### SUMMARY OF THE INVENTION

### Problems to be Solved by the Invention

Fluorescent probes of the above prior art succeeded in imaging sulfane sulfur in cultured cells, but experiments are conducted with a large amount of surfactant added because both sides of the xanthene cyclic phenolic hydroxyl group of fluorescein are modified, and the water solubility is very poor due to that molecular structure. These are also an irreversible type of fluorescent probe that releases a fluorophore, and there appears to be room for improvement to application to living cells.

The purpose of the present invention is to provide a novel fluorescent probe that shows sufficient solubility in water and detects sulfane sulfur in vivo.

### Means for Solving the Problems

The present inventors conducted in-depth studies intended to design a novel fluorescent dye having a structure wherein a dye molecule as a donor capable of causing FRET (fluorescence resonance energy transfer) is bonded to a fluorescent dye having a xanthene skeleton as a mother nucleus and further, fluorescence derived from the xanthene dye is not emitted after reaction with sulfane sulfur. They discovered that the above problems can be solved by introducing specific substituents into the benzene ring bonded to the xanthene skeleton and completed the present invention.

Specifically, the present invention provides:

[1] A compound of the following general formula (I):

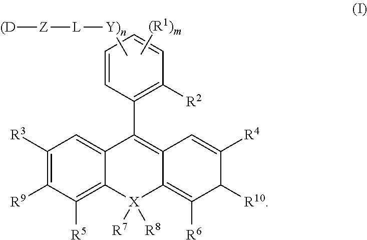

wherein,

R¹ is a hydrogen atom or the same or different monovalent substituents present on the benzene ring;
R² is SH or S—S—R (R is a C₁₋₆alkyl group);
R³ and R⁴ are, each independently, a hydrogen atom, C₁₋₆ alkyl group, or halogen atom;
R⁵ and R⁶ are, each independently, a hydrogen atom, C₁₋₆alkyl group, or halogen atom;
R⁷ and R⁸ are, when present, each independently a C₁₋₆ alkyl group or aryl group;
wherein, when X is an oxygen atom, R⁷ and R⁸ are not present, when X is a phosphorus atom, one of —R⁷ and —R⁸ may be ═O;
R⁹ and R¹⁰

(i) are each independently selected from NH₂, monoalkylamino groups, or dialkylamino groups, or

(ii) are each independently selected from a hydroxyl group or alkoxy group;

X is an oxygen atom, silicon atom, tin atom, germanium atom, or phosphorus atom;
Y is a linking group a with L;
L is a linker;
Z is a linking group b with L;
D is fluorescein, a fluorescein derivative, coumarin, a coumarin derivative, rhodamine, or a rhodamine derivative;
m is an integer of 0-3, n is an integer of 1-4, and m+n=4; or a salt thereof.

[2] The compound according to [1] represented by the following general formula (Ia):

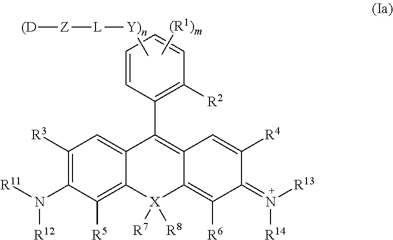

wherein,

R¹-R⁸, X, Y, L, Z, D, m, and n are as defined in general formula (I);

R¹¹ and R¹² are, each independently, a hydrogen atom or C₁₋₆ alkyl group,

R¹¹ or R¹², together with R³ or R⁵, may form a five- to seven-membered heterocycle or heteroaryl containing the nitrogen atoms to which R¹¹ or R¹² is bonded, may also contain from one to three heteroatoms selected from the group consisting of an oxygen atom, nitrogen atom, or sulfur atom as ring members, and the heterocycle or heteroaryl may also be substituted by C₁₋₆alkyl, C₂₋₆ alkenyl, or C₂₋₆ alkynyl, C₆₋₁₀ aralkyl group, or C₆₋₁₀ alkyl-substituted alkenyl group; R¹³ and R¹⁴ are, each independently, a hydrogen atom or C₁₋₆ alkyl group,

R¹³ or R¹⁴, together with R⁴ or R⁶, may form a five- to seven-membered heterocycle or heteroaryl containing the nitrogen atoms to which R¹³ or R¹⁴ is bonded, may also contain from one to three heteroatoms selected from the group consisting of an oxygen atom, nitrogen atom, or sulfur atom as ring members, and the heterocycle or heteroaryl may also be substituted by C₁₋₆ alkyl, C₂₋₆ alkenyl, or C₂₋₆ alkynyl, C₆₋₁₀ aralkyl group, or C₆₋₁₀ alkyl-substituted alkenyl group; or a salt thereof.

[3] The compound according to [1] represented by the following general formula (Ib):

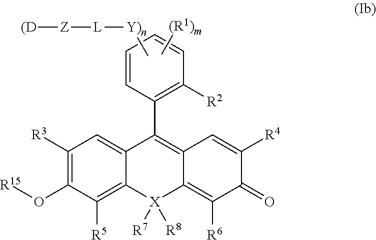

wherein,

R¹-R⁸, X, Y, L, Z, D, m, and n are as defined in general formula (I);
R¹⁵ is a hydrogen atom or C₁₋₆ alkyl group;
or a salt thereof.

[4] The compound according to any one of [1]-[3] wherein the linker is selected from a cycloalkyl group or aryl group, or a salt thereof.

[5] The compound according to any one of [1]-[4] wherein the linking group a is selected from a carbonyl group, alkylcarbonyl group, ester group, alkyl ester group, amino group, alkylamino group, amide group, alkylamide group, isothiocyanate group, sulfonyl chloride group, haloalkyl group, haloacetamide group, azide group, or alkynyl group, or a salt thereof.

[6] The compound according to any one of [1]-[5] wherein the linking group b is selected from a carbonyl group, alkylcarbonyl group, ester group, alkyl ester group, amino group, alkylamino group, amide group, alkylamide group, isothiocyanate group, sulfonyl chloride group, haloalkyl group, haloacetamide group, azide group, or alkynyl group, or a salt thereof.

[7] A fluorescent probe containing a compound according to any one of [1]-[6] or a salt thereof.

[8] A method for detecting sulfane sulfur in cells comprising;

(a) a step for introducing a compound according to any one of [1]-[6] or a salt thereof into cells and

(b) a step for measuring the fluorescence emitted by the compound or salt thereof in the cells.

### Advantages of the Invention

The present invention makes it possible to provide a fluorescent probe that shows sufficient solubility in water and detects sulfane sulfur in vivo.

In particular, the present invention makes it possible to provide an excellent OFF/ON type of fluorescent probe because fluorescence is not emitted from the donor dye molecule due to the FRET effect prior to reaction with sulfane sulfur, but strong fluorescence from the donor dye molecule is emitted after reaction.

In addition, the fluorescent probe of the present invention can also be used in ratio measurement in combination with a decrease in the fluorescence of the fluorescent dye having a xanthene skeleton as a mother nucleus that serves as an acceptor because fluorescence is not emitted from the donor dye molecule prior to reaction with sulfane sulfur, but strong fluorescence from the donor dye molecule is emitted after reaction.

### BRIEF DESCRIPTION OF THE DRAWINGS

FIG. 1 is a schematic drawing of the production of reactive sulfur species by enzyme and the selective detection thereof.

FIG. 2 is a schematic drawing of fluorescent imaging methods of the prior art.

FIG. 3 is the results of measurement of the absorption spectrum and the fluorescence spectrum after adding Na₂S₄ to compound 8 (SSip-1).

FIG. 4 is the results of measurement of the absorption spectrum and the fluorescence spectrum of 1 μM of SSip-1 in 0.1 M sodium phosphate buffer in the presence of 5 mM of GSH after addition of 50 μM of Na₂S₄.

FIG. 5 is the results obtained by studying the selectivity of SSip-1 with H₂S.

FIG. 6 is the results of imaging of live cells (A549 cells) by SSip-1;

FIG. 7 is the results of measurement of the absorption spectrum and fluorescence spectrum before and after addition of Na₂S₄ to compound a1.

FIG. 8 is the results of measurement of the absorption spectrum and fluorescence spectrum before and after addition of Na₂S₄ to compound a2.

FIG. 9 is the results of measurement of the absorption spectrum and fluorescence spectrum before and after addition of Na₂S₄ to compound 25.

FIG. 10 is confocal microscope images of live A549 cells using SSip-1 with 250 μM of Na₂S₄ added.

FIG. 11 is the results of observation (confocal microscope images) of the reversibility of SSip-1 in live cells.

FIG. 12 is the results of observation (changes in fluorescence intensity) of the reversibility of SSip-1 in live cells.

FIG. 13 is the changes in the ratio (FL/RB) of live A549 cells incubated by SSip-1 after repeated addition of 250 μM of Na₂S₄ and ratio images thereof.

FIG. 14 is the results of co-staining with SSip-1 and Lysotracker or Mitrotracker.

FIG. 15 is confocal microscope images of live A549 cells using compound a4 with 250 μM of Na₂S₄ added.

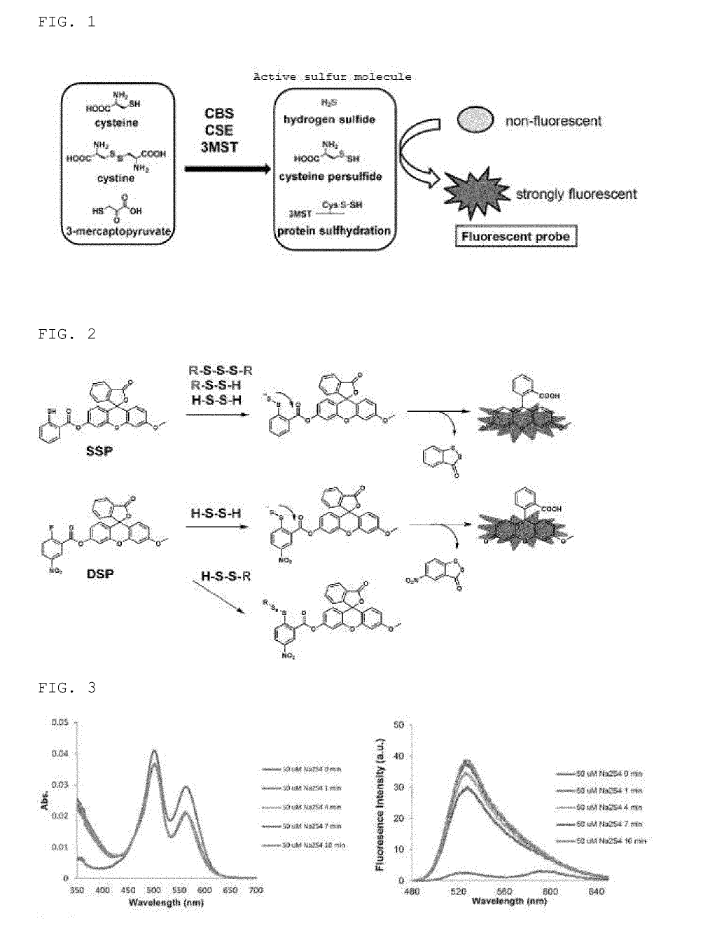

### BEST MODE FOR CARRYING OUT THE INVENTION

In the present specification, an “alkyl group” or alkyl moiety of a substituent including an alkyl moiety (such as an alkoxy group), when not mentioned in particular, means a C₁₋₆, preferably C₁₋₄, more preferably C₁₋₃, linear, branched, cyclic, or combination thereof alkyl group. More specific examples include a methyl group, ethyl group, n-propyl group, isopropyl group, cyclopropyl group, n-butyl group, sec-butyl group, isobutyl group, tert-butyl group, cyclopropylmethyl group, n-pentyl group, n-hexyl group, and the like as alkyl groups.

When “halogen atom” is stated in the present specification, it may be any of a fluorine atom, chlorine atom, bromine atom, or iodine atom, preferably a fluorine atom, chlorine atom, or bromine atom.

One embodiment of the present invention is a compound represented by the following general formula (I) or a salt thereof.

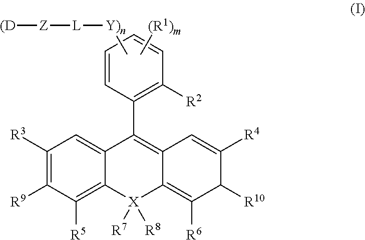

In general formula (I), R¹ is a hydrogen atom or the same or different monovalent substituents present on the benzene ring.

When R¹ represents monovalent substituents present on the benzene ring, one or two of the same or different substituents are preferably present on the benzene ring. When R¹ represents one or more monovalent substituents, the substituents can be substituted at any position on the benzene ring. Preferably, all R¹ are hydrogen atoms, or one R¹ is a monovalent substituent and the other R¹ are hydrogen atoms.

The type of monovalent substituent represented by R¹ is not particularly restricted, but monovalent substituents are preferably selected from the group consisting of a C₁₋₆ alkyl group, C₁₋₆ alkenyl group, C₁₋₆ alkynyl group, C₁₋₆ alkoxy groups, hydroxyl group, carboxyl group, sulfonyl group, alkoxycarbonyl group, halogen atom, or amino group. These monovalent substituents may also have one or more arbitrary substituents.

For example, one or more halogen atoms, carboxy groups, sulfonyl groups, hydroxyl groups, amino groups, alkoxy groups, and the like may be present in alkyl groups represented by R¹, and alkyl groups represented by R¹ may be alkyl halide groups, hydroxyalkyl groups, carboxyalkyl groups, aminoalkyl groups, or the like. One or more alkyl groups may also be present in amino groups represented by R¹, and amino groups represented by R¹ may be monoalkylamino groups or dialkylamino groups. In addition, carboxy-substituted alkoxy groups, alkoxycarbonyl-substituted alkoxy groups, and the like can be given as examples of when alkoxy groups represented by R¹ have substituents; more specific examples include a 4-carboxybutoxy group, 4-acetoxymethyloxycarbonylbutoxy group, and the like.

In one preferred aspect, R¹ is a monovalent substituent such as a C₁₋₆ alkyl group, and the substituent is present at from position 3 to position 6 on the benzene ring.

R² is SH or S—S—R (R represents a C₁₋₆, preferably C₁₋₂, alkyl group).

Though not intending to be bound by theory, an OFF/ON type of probe can be provided because, since a donor dye molecule capable of causing FRET is introduced as D in a compound of general formula (I), fluorescence derived from the donor dye is not emitted prior to sulfane sulfur addition, but the fluorescence intensity derived from D rises because FRET does not occur due to formation of a closed ring structure by the reaction of sulfane sulfur and the SH or S—S—R of R² when sulfane sulfur is added. The introduction of SH or S—S—R at the position of R² is therefore important in a compound of general formula (I).

In general formula (I), R³ and R⁴ are, each independently, a hydrogen atom, C₁₋₆ alkyl group, or halogen atom. When R³ or R⁴ is an alkyl group, one or more halogen atoms, carboxy groups, sulfonyl groups, hydroxyl groups, amino groups, alkoxy groups, or the like may be present in the alkyl group. For example, an alkyl group represented by R³ or R⁴ may be an alkyl halide group, hydroxyalkyl group, carboxyalkyl group, or the like. R³ and R⁴ are preferably each independently a hydrogen atom or halogen atom; more preferred is when both R³ and R⁴ are hydrogen atoms or both R³ and R⁴ are fluorine atoms or chlorine atoms.

In general formula (I), R⁵ and R⁶ are, each independently, a hydrogen atom, C₁₋₆ alkyl group, or halogen atom, the explanation of which is the same as for R³ and R⁴. It is preferred that R⁵ and R⁶ are both hydrogen atoms, or both chlorine atoms, or both fluorine atoms.

In general formula (I), R⁷ and R⁸ are, when present, each independently a C₁₋₆ alkyl group or aryl group, but R⁷ and R⁸, each independently, are preferably C₁₋₃ alkyl groups, and R⁷ and R⁸ are more preferably both methyl groups. One or more halogen atoms, carboxy groups, sulfonyl groups, hydroxyl groups, amino groups, alkoxy groups, or the like may be present in an alkyl group represented by R⁷ and R⁸; for example, alkyl groups represented by R⁷ and R⁸ may be alkyl halide groups, hydroxyalkyl groups, carboxyalkyl groups, or the like.

When R⁷ or R⁸ is an aryl group, the aryl group may be a monocyclic aromatic group or a condensed aromatic group; and the aryl ring may include one or more ring member heteroatoms (such as a nitrogen atom, oxygen atom, or sulfur atom). A phenyl group is preferred as the aryl group. One or more substituents may be present on the aryl ring. For example, one or more halogen atoms, carboxy groups, sulfonyl groups, hydroxyl groups, amino groups, alkoxy groups, or the like may be present as substituents.

When X, which will be described later, is an oxygen atom, R⁷ and R⁸ are not present.

When X is a phosphorus atom, one of —R⁷ and —R⁸ may be ═O. In a preferred aspect of when X is a phosphorus atom, one of —R⁵ and —R⁶ [sic] is ═O and the other represents a C₁₋₆ alkyl group or aryl group.

As R⁹ and R¹⁰, there is (i) an embodiment whereby each is independently selected from NH₂, monoalkylamino groups, or dialkylamino groups or (ii) an embodiment whereby each is independently selected from a hydroxyl group or alkoxy group.

The monoalkylamino groups and dialkylamino groups preferably have C₁₋₆ substituted or unsubstituted alkyl groups. Examples of substituents include a methyl group, ethyl group, ethylcarboxy group, and the like.

The alkoxy group is preferably a C₁₋₆ alkoxy group, more preferably a methoxy group or ethoxy group.

In the above embodiment (i), when at least one of R⁹ and R¹⁰ is a monoalkylamino group or dialkylamino group, the monoalkylamino group or dialkylamino group, together with any of R⁴-R⁶, may form a five- to seven-membered heterocycle or heteroaryl containing the nitrogen atoms of the monoalkylamino group or dialkylamino group, may also contain one to three heteroatoms selected from the group consisting of an oxygen atom, nitrogen atom, and sulfur atom as ring members, and the heterocycle or heteroaryl may also be substituted by C₁₋₆ alkyl, C₂₋₆ alkenyl, or C₂₋₆ alkynyl, C₆₋₁₀ aralkyl group, or C₆₋₁₀ alkyl-substituted alkenyl group.

In general formula (I), X is an oxygen atom, silicon atom, tin atom, germanium atom, or phosphorus atom. In a preferred embodiment of the present invention, X is an oxygen atom.

Y represents a linking group a for coupling the benzene ring and L. Examples of the linking group a include a carbonyl group, alkylcarbonyl group, ester group, alkyl ester group, amino group, alkylamino group, amide group, alkylamide group, isothiocyanate group, sulfonyl chloride group, haloalkyl group, haloacetamide group, azide group, alkynyl group, and the like.

A carbonyl group, amide group, and alkylamide group are especially preferred.

L represents a linker. The linker is selected from structurally rigid structures that prevent the fluorescent dye having a xanthene skeleton as the mother nucleus from coming close to the dye molecule as the donor that can cause FRET.

The linker is preferably a cycloalkyl group or aryl group.

A trans-cyclohexyl group is preferred as the cycloalkyl group. A phenyl group is preferred as the aryl group.

Z represents a linking group b for coupling L and D. Examples of the linking group b include a carbonyl group, alkylcarbonyl group, ester group, alkyl ester group, amino group, alkyl amino group, amide group, alkylamide group, isothiocyanate group, sulfonyl chloride group, haloalkyl group, haloacetamide group, azide group, alkynyl group, and the like. A carbonyl group, amide group, and alkylamide group are especially preferred.

D represents a donor dye that triggers a FRET phenomenon on the xanthene dye that is the mother nucleus. Preferred examples of such a dye include fluorescein, fluorescein derivatives, coumarin, coumarin derivatives, rhodamine, and rhodamine derivatives.

Examples of fluorescein derivatives include fluorescein having substituents, fluorescein derivatives in which a hydroxyl group of the xanthene skeleton has been acetylated (said derivatives may have other substituents), fluorescein derivatives in which the COOH on the phenyl group bonded to the xanthene skeleton forms a closed-ring structure with the xanthene skeleton (said derivatives may have other substituents), and the like. An alkylamine having a carboxyl group, alkylamine having a carboxy ester group (for example, —COOCH₂OCOCH₃), chloro group, fluoro group, methyl group, and the like are preferred as substituents. The positions at which substituents are introduced are not particularly restricted, but positions 2, 4, 5, and 7 of the xanthene ring are preferred.

Coumarin derivatives are coumarin having substituents. A hydroxy group, chlorine atom, acetoxy group, alkoxy group having an acetoxy group, trifluoromethyl group, and the like are preferred as substituents. The positions at which substituents are introduced are preferably positions 3, 4, and 6 of coumarin.

Examples of rhodamine derivatives include rhodamine having substituents, rhodamine derivatives in which an amino group of the xanthene skeleton has been alkylated (said derivatives may have other substituents), rhodamine derivatives in which the COOH on the phenyl group bonded to the xanthene skeleton forms a closed-ring structure with the xanthene skeleton (said derivatives may have other substituents), and the like. A chloro group, fluoro group, and the like are preferred as substituents.

Non-limiting examples of fluorescein and fluorescein derivatives include the following.

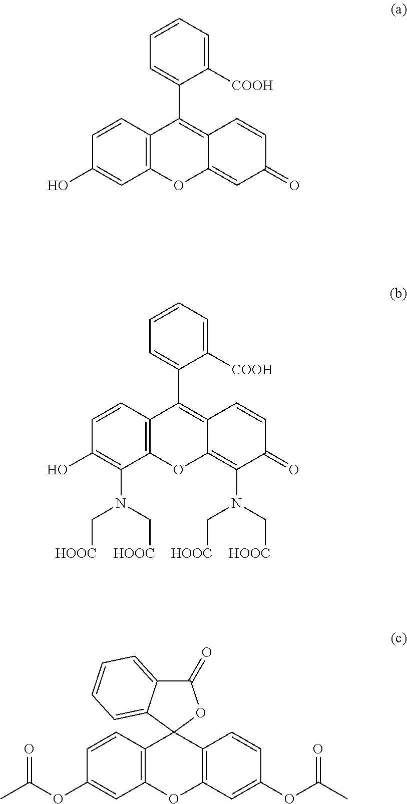 [SMILES: O=C(O)c1ccccc1-c1c2ccc(=O)cc-2oc2cc(O)ccc12 ; O=C(O)CN(CC(=O)O)c1c2oc3c(N(CC(=O)O)CC(=O)O)c(O)ccc3c(-c3ccccc3C(=O)O)c-2ccc1=O ; CC(=O)Oc1ccc2c(c1)Oc1cc(OC(C)=O)ccc1C21OC(=O)c2ccccc21]

Non-limiting examples of coumarin and coumarin derivatives include the following.

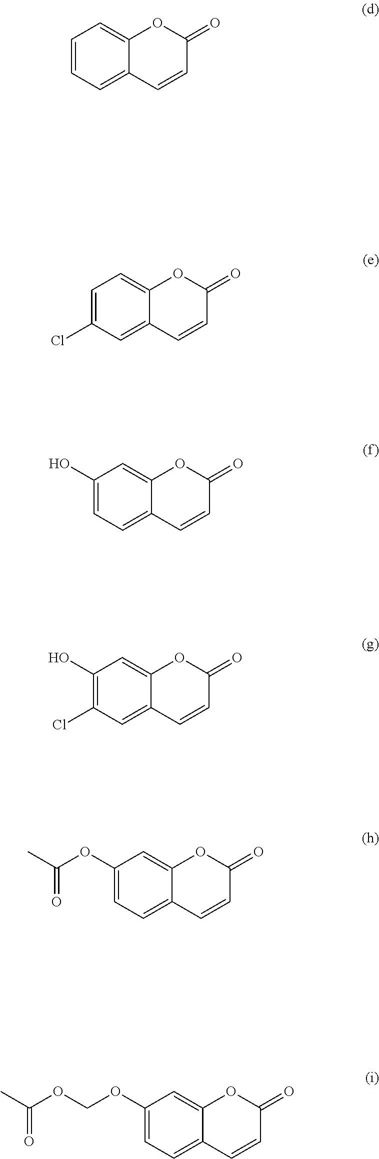 [SMILES: O=c1ccc2ccccc2o1 ; O=c1ccc2cc(Cl)ccc2o1 ; O=c1ccc2ccc(O)cc2o1 ; O=c1ccc2cc(Cl)c(O)cc2o1 ; CC(=O)Oc1ccc2ccc(=O)oc2c1 ; CC(=O)OCOc1ccc2ccc(=O)oc2c1]

When fluorescein or a fluorescein derivative is connected to a linker via a linking group b, the linking group b is preferably introduced at position 4 or 5 on the phenyl group bonded to the xanthene skeleton of the fluorescein or fluorescein derivative.

When coumarin or a coumarin derivative is connected to a linker via a linking group b, the linking group b is preferably introduced at position 3 or 4 of the coumarin or coumarin derivative.

Non-limiting examples of when a coumarin derivative is connected to a linker appear below.

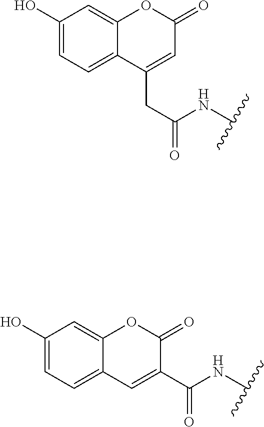 [SMILES: *NC(=O)Cc1cc(=O)oc2cc(O)ccc12 ; *NC(=O)c1cc2ccc(O)cc2oc1=O]

In general formula (I), m is an integer of 0-3, n is an integer of 1-4, and m+n=4.

In a preferred embodiment of the present invention, m is 3 and n is 1.

One aspect of the present invention is a compound represented by the following general formula (Ia) or a salt thereof.

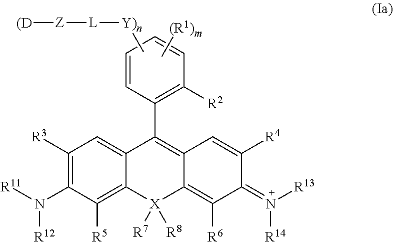

In general formula (Ia), R¹-R⁸, X, Y, L, Z, D, m, and n are as defined in general formula (I).

In general formula (Ia), R¹¹ and R¹² are, each independently, a hydrogen atom or C₁₋₆ alkyl group.

R¹¹ or R¹², together with R³ or R⁵, may form a five- to seven-membered heterocycle or heteroaryl containing the nitrogen atoms to which R¹¹ or R¹² is bonded, may also contain from one to three heteroatoms selected from the group consisting of an oxygen atom, nitrogen atom, or sulfur atom as ring members, and the heterocycle or heteroaryl may also be substituted by C₁₋₆ alkyl, C₂₋₆ alkenyl, or C₂₋₆ alkynyl, C₆₋₁₀ aralkyl group, or C₆₋₁₀ alkyl-substituted alkenyl group.

Preferably, R¹¹ and R¹² each independently are a hydrogen atom, methyl group, or ethyl group.

In general formula (Ia), R¹³ and R¹⁴ each independently represent a hydrogen atom or C₁₋₆ alkyl group.

R¹³ or R¹⁴, together with R⁴ or R⁶, may form a five- to seven-membered heterocycle or heteroaryl containing the nitrogen atoms to which R¹³ or R¹⁴ is bonded, may also contain from one to three heteroatoms selected from the group consisting of an oxygen atom, nitrogen atom, or sulfur atom as ring members, and the heterocycle or heteroaryl may also be substituted by C₁₋₆ alkyl, C₂₋₆ alkenyl, or C₂₋₆ alkynyl, C₆₋₁₀ aralkyl group, or C₆₋₁₀ alkyl-substituted alkenyl group.

Preferably, R¹³ and R¹⁴ each independently are a hydrogen atom, methyl group, or ethyl group.

One aspect of the present invention is a compound represented by the following general formula (Ib) or a salt thereof.

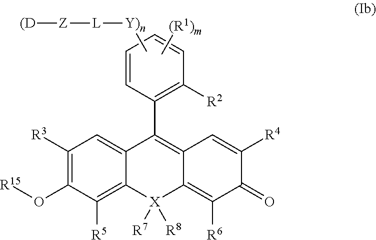

In general formula (Ib), R¹-R⁸, X, Y, L, Z, D, m, and n are as defined in general formula (I).

In general formula (Ib), R¹⁵ is a hydrogen atom or C₁₋₆ alkyl group.

Preferably, R¹⁵ is a hydrogen atom, methyl group, or ethyl group.

Non-limiting examples of compounds included in general formulas (I), (Ia), and (Ib) appear below.

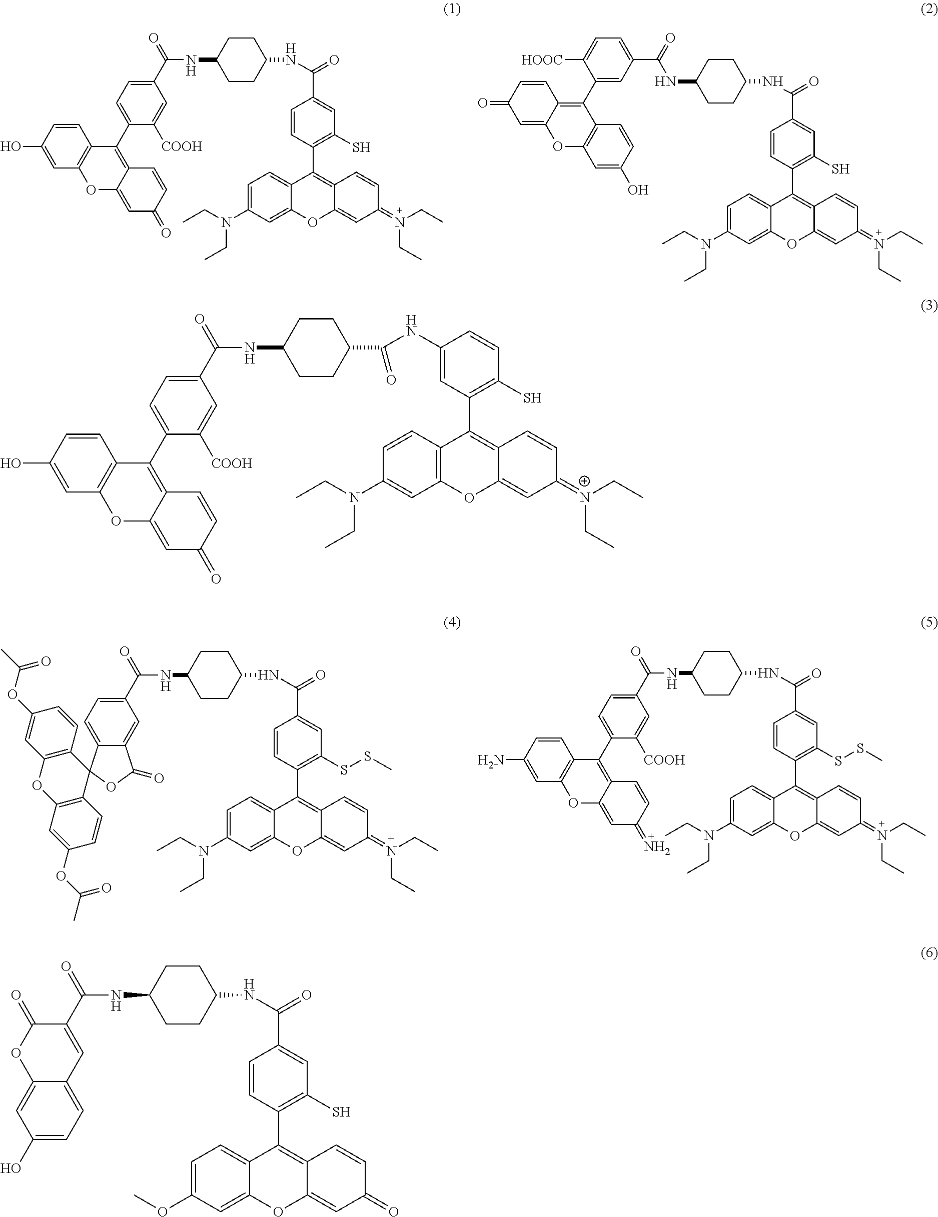

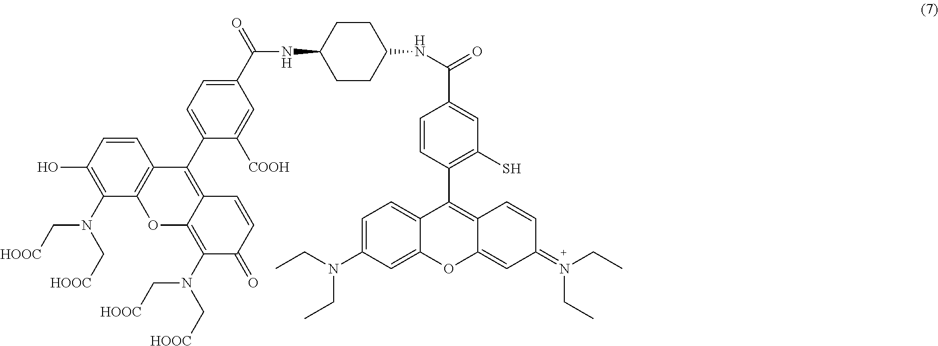

Compounds of the present invention represented by formulas (I), (Ia), and (Ib) can exist as acid addition salts or base addition salts. Examples of acid addition salts can include mineral acid salts such as a hydrochloride, sulfate, nitrate, and the like or organic acid salts such as a methanesulfonate, p-toluenesulfonate, oxalate, citrate, tartrate, and the like. Examples of base addition salts can include metal salts such as a sodium salt, potassium salt, calcium salt, magnesium salt, and the like, an ammonium salt, or an organic amine salt such as a triethylamine salt, and the like. In addition to these, there are also cases in which a salt is formed with an amino acid such as glycine. Compounds of the present invention or salts thereof can also sometimes exist as hydrates or solvates. These substances are also within the scope of the present invention.

Compounds of the present invention represented by formulas (I), (Ia), and (Ib) sometimes have one or more asymmetrical carbons, depending on the types of substituents. Stereoisomers such as optically active compounds based on one or more asymmetrical carbons and diastereomers based on two or more asymmetrical carbons as well as any mixtures of stereoisomers, racemates, and the like are all encompassed within the scope of the present invention.

Typical compound production methods for compounds of the present invention are shown concretely in the examples in this specification. Therefore, one skilled in the art can produce compounds of the present invention represented by general formulas (I), (Ia), and (Ib) by appropriately selecting the reaction raw materials, reaction conditions, reaction reagents, and the like and by modifying or changing these methods as needed.

Compounds of the present invention represented by formulas (I), (Ia), and (Ib) of the present invention are useful as fluorescent probes for detecting sulfane sulfur in cells.

Specifically, another embodiment of the present invention is a fluorescent probe including a compound represented by formula (I) or a salt thereof.

Another embodiment of the present invention is a method for detecting sulfane sulfur in cells wherein the method includes (a) a step for introducing a compound represented by formula (I), (Ia), or (Ib) or a salt thereof into cells and (b) a step for measuring the fluorescence emitted by the compound or salt thereof into the cells.

Compounds of the present invention represented by formulas (I), (Ia), and (Ib) or salts thereof characteristically are substantially non-fluorescent or have only weak fluorescence in environments free of sulfane sulfur, but emit strong fluorescence derived from the donor dye in environments where sulfane sulfur is present. Therefore, compounds of the present invention represented by formulas (I), (Ia), and (Ib) or salts thereof are highly useful in that they make it possible to provide an OFF/ON type of fluorescent probe for detecting sulfane sulfur in cells under physiological conditions.

The method for using a fluorescent probe of the present invention is not particularly restricted, and the probe can be used in the same way as conventional, known fluorescent probes. A compound represented by the above formula (I), (Ia), and (Ib) is usually dissolved in an aqueous medium such as physiological saline, a buffer, or the like or a mixture of an aqueous medium and a water-miscible organic solvent such as ethanol, acetone, ethylene glycol, dimethylsulfoxide, dimethylformamide, or the like. This solution is added to a suitable buffer that includes cells or tissues, and the fluorescence spectrum may be measured. The fluorescent probe of the present invention may also be used in the form of a composition in combination with suitable additives. For example, it can be combined with additives such as buffers, dissolution auxiliaries, pH regulators, and the like.

### Examples

The present invention is explained below through examples. The present invention, however, is not limited to these examples.

### Synthesis Example 1

### Synthesis of Compound 8 (SSip-1) of the Present Invention

A synthesis intermediate for use in synthesizing compound 8 of the present invention was synthesized according to scheme 1 below.

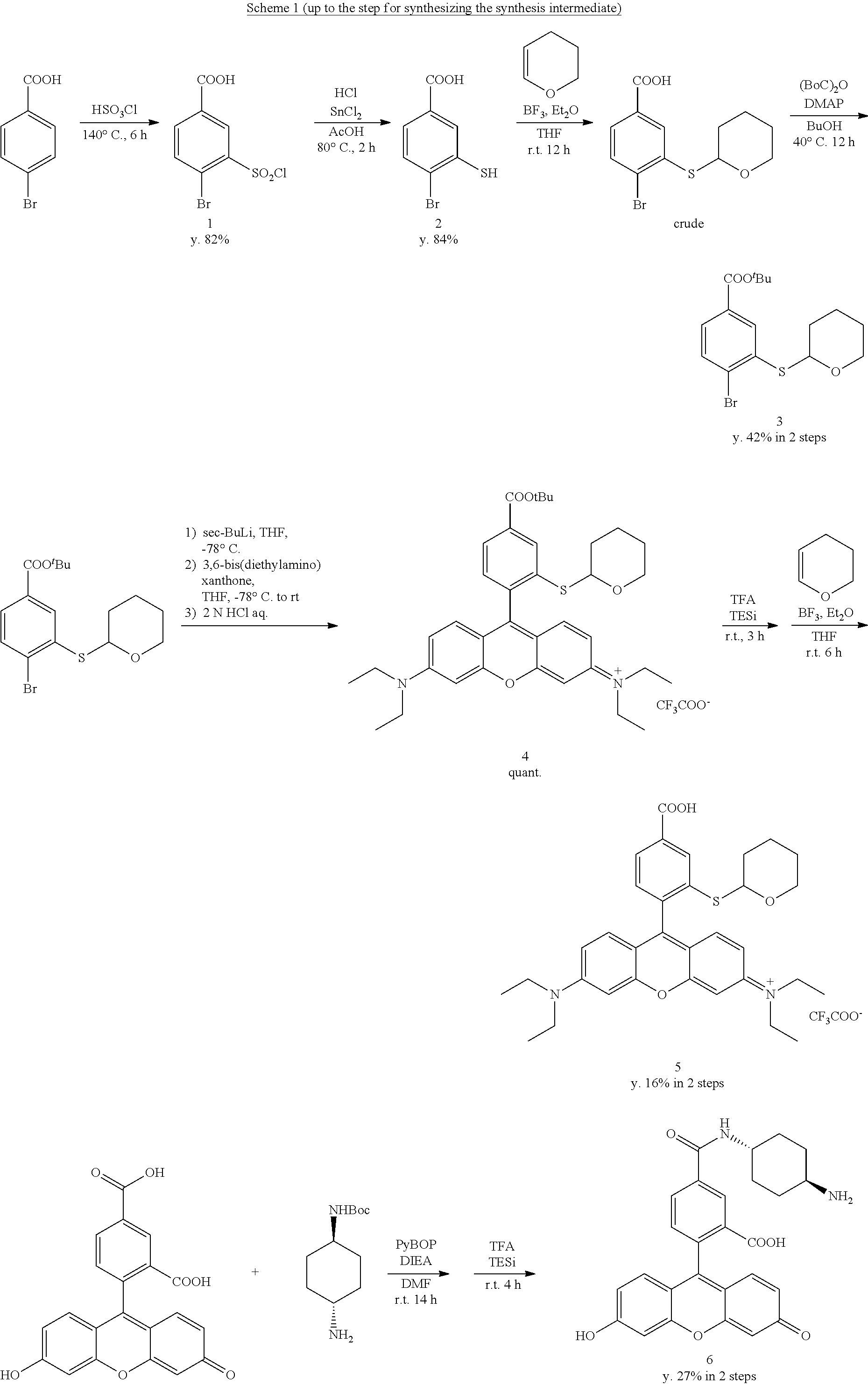

### (1) Synthesis of Compound 1

4-Bromobenzoic acid (5.06 g, 25.3 mmol) was placed in a flask, and chlorosulfonic acid (12 mL) was slowly added dropwise. The solution was heated under reflux for six hours at 140° C. After cooling to room temperature, the reaction solution was slowly added dropwise to ice water. A precipitate of the target substance was produced at that time. The precipitate was collected by a Buchner funnel, the funnel was washed by H₂O, and 4-bromo-3-chlorosulfonylbenzoic acid (compound 1, 6.17 g, 20.7 mmol, yield 82%) was obtained. ¹H NMR (400 MHz, Acetone-d): δ 8.23 (d, 1H, J=8.3 Hz), 8.33 (dd, 1H, J=8.3, 2.0 Hz), 8.74 (d, 1H, J=2.0 Hz.) ¹³C NMR (100 μMHz, Acetone-d₆): δ 125.8, 132.1, 132.2, 137.8, 138.4, 143.8, 165.0. HRMS (ESI⁻): Calcd for [M−H]⁻ 298.8604, Found 298.8589 (−1.5 mmu).

### (2) Synthesis of Compound 2

4-Bromo-3-chlorosulfonylbenzoic acid (compound 1, 6.17 g, 20.7 mmol) was dissolved in acetic acid (60 mL), and tin(II) chloride (27.8 gm, 123.6 mmol) dissolved in 10N HCl aq. (25 mL) was added thereto and stirred for two hours at 80° C. in an argon atmosphere. After cooling to room temperature, the precipitate of the target substance produced was collected by a Buchner funnel, and funnel was washed by H₂O, and 4-bromo-3-mercaptobenzoic acid (compound 2, 4.03 g, 17.0 mmol, yield 84%) was obtained.

¹H NMR (400 μMHz, Acetone-d): δ 5.04 (1H, s), 7.66 (dd, 1H, J=8.3, 2.0 Hz), 7.72 (d, 1H, J=8.3 Hz), 8.14 (d, 1H, J=2.0 Hz) ¹³C NMR (100 μMHz, Acetone-d₆): δ 126.7, 128.2, 131.4, 131.5, 134.0, 136.7, 166.5.

HRMS (ESI⁻): Calcd for [M−H]⁻ 232.9095, Found 232.9139 (−4.4 mmu).

### (3) Synthesis of Compound 3

4-Bromo-3-mercaptobenzoic acid (compound 2, 500 mg, 2.20 mmol) and 3,4-dihydro-2H-pyran (360 mg, 4.3 mmol) were dissolved in dehydrated tetrahydrofuran (20 mL) and cooled to 0° C. in an argon atmosphere. A boron trifluoride-ethyl ether complex (312 mg, 2.20 mmol) was added thereto and stirred for 12 hours at room temperature. The solvent was dissolved off under reduced pressure, and water was added to the residue. This mixture was extracted by ethyl acetate and washed by brine. The organic layer was dried by Na₂SO₄ and the solvent was distilled off under reduced pressure. Some of the byproducts were then removed by column chromatography (silica gel, ethyl acetate/n-hexane).

The crudely purified compound was dissolved in tert-butyl alcohol (15 mL), and 4-dimethylaminopyridine (38 mg, 0.31 mmol) and di-tert-butyl dicarbonate (239 mg, 1.10 mmol) were added and stirred for 12 hours at 40° C. in an argon atmosphere. The solvent was distilled off under reduced pressure, and water was added to the residue. This mixture was extracted by ethyl acetate and washed by brine. The organic layer was dried by Na₂SO₄ and the solvent was distilled off under reduced pressure. Tert-butyl 4-bromo-3-(tetrahydropyran-2-ylthio)benzoate (compound 3, 338 mg, 0.91 mmol, yield 42%) was obtained by column chromatography (silica gel, dichloromethane/n-hexane).

¹H NMR (300 μMHz, CDCl₃): δ 1.59 (s, 9H), 1.64-1.77 (m, 3H), 1.87-1.98 (m, 2H), 2.05-2.13 (m, 1H), 3.63-3.71 (m, 1H), 4.13-4.20 (m, 1H), 5.35-5.38 (m, 1H), 7.56 (d, 1H, J=8.1 Hz), 7.62 (dd, 1H, J=1.8, 8.4 Hz), 8.19. (d, 1H, J=1.5 Hz) ¹³C NMR (75 μMHz, CDCl₃): δ 21.5, 25.2, 28.0, 31.0, 64.6, 81.3, 83.6, 127.5, 128.0, 130.2, 131.6, 132.4, 137.6, 164.6.

### (4) Synthesis of Compound 4

Tert-butyl 4-bromo-3-(tetrahydropyran-2-ylthio)benzoate (compound 3, 600 mg, 1.61 mmol) and dehydrated tetrahydrofuran (15 mL) were added to a dried, argon-purged flask. After cooling to −78° C., 1 M sec-butyllithium (1.0 mL, 1.0 mmol) was added and stirred for 20 minutes. 3,6-Bis(diethylamino)xanthone (100 mg, 0.30 mmol) was dissolved in dehydrated tetrahydrofuran (5 mL) and slowly added at the same temperature, and the mixture was returned to room temperature and stirred for one hour. The reaction was stopped by 2N HCl aq. This mixture was extracted by dichloromethane and washed by brine. The organic layer was dried by Na₂SO₄, and the solvent was then distilled off under reduced pressure. The residue was purified by HPLC (eluent, from 20% acetonitrile/0.1% trifluoroacetic acid/water (0 min) to 80% acetonitrile/0.1% trifluoroacetic acid/water (15 min); flow rate=25.0 mL/min), and 2-S-THP-4-tert-butoxycarbonyl-tetraethylrhodamine (compound 4, 190 mg, 0.30 mmol, quant.) was obtained.

¹H NMR (400 μMHz, CD₃OD): δ 1.32 (t, 12H, J=6.8 Hz), 1.43-1.62 (m, 4H), 1.65 (s, 1H), 1.82-1.85 (m, 2H), 3.47-3.52 (m, 1H), 3.68-3.72 (m, 8H), 3.86-3.89 (m, 1H), 5.27-5.29 (m, 1H), 7.00 (d, 2H, J=2.4 Hz), 7.06-7.09 (m, 2H), 7.15 (dd, 1H, J=2.2, 9.5 Hz), 7.42 (d, 1H, J=7.8 Hz), 8.05 (dd, 1H, J=1.7, 8.1 Hz), 8.48 (d, 1H, J=1.5 Hz) ¹³C NMR (75 MHz, CD₃OD): δ 12.8, 22.4, 26.4, 28.4, 32.4, 46.9, 65.6, 83.2, 86.4, 97.4, 114.4, 115.7, 128.5, 131.0, 132.5, 132.9, 135.3, 137.7, 138.4, 156.7, 157.4, 159.4, 166.1. HRMS (ESI⁺): Calcd for [M]⁺ 615.3257, Found 615.3223 (−3.4 mmu).

### (5) Synthesis of Compound 5

Trifluoroacetic acid (3 mL) and triethylsilane (20 μL) were added to compound 4 (20 mg, 0.033 mmol) and stirred for three hours at room temperature. After distilling off the solvent under reduced pressure, some of the byproducts were removed by subjecting the residue to HPLC (eluent, from 20% acetonitrile/0.1% trifluoroacetic acid/water (0 min) to 80% acetonitrile/0.1% trifluoroacetic acid/water (15 min); flow rate=25.0 mL/min).

The crudely purified compound was dissolved in THF (5 mL), and 3,4-dihydro-2H-pyran (6 mg, 0.07 mmol) and a boron trifluoride-ethyl ether complex (10 mg, 0.07 mmol) were added thereto and stirred for six hours at room temperature. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=25.0 mL/min), and 2-S-THP-4-carboxytetraethylrhodamine (compound 5, 3.3 mg, 0.0059 mmol, yield 18%) was obtained.

¹H NMR (300 μMHz, CD₃OD): δ 1.32 (t, 12H, J=7.0 Hz), 1.49-1.58 (m, 5H), 1.86-1.99 (m, 1H), 3.45-3.53 (m, 1H), 3.70 (q, 8H, J=6.8 Hz), 3.84-3.88 (m, 1H), 5.33 (t, 1H, J=4.4 Hz), 7.01 (d, 2H, J=3.0 Hz), 7.08 (d, 2H, J=10.2 Hz), 7.17 (dd, 1H, J=3.6, 9.6 Hz), 7.44 (d, 1H, J=8.1 Hz), 8.12 (dd, 1H, J=1.5, 8.1 Hz), 8.53 (d, 1H, J=1.5 Hz) ¹³C NMR (100 μMHz, CD₃OD): δ 12.8, 22.2, 26.4, 32.5, 46.9, 65.3, 86.5, 97.5, 114.6, 115.7, 128.9, 131.1, 132.8, 133.4, 134.4, 137.7, 138.8, 156.7, 157.4, 159.4, 168.4. HRMS (ESI⁺): Calcd For [M+H]⁺ 559.2631, Found 559.2593 (−3.8 mmu).

### (6) Synthesis of Compound 6

5-Carboxyfluorescein (36 mg, 0.096 mmol) and N-Boc-trans-1,4-cyclohexanediamine (36 mg, 0.17 mmol) and N,N-diisopropylethylamine (65 mg, 0.50 mmol) and 1H-benzotriazol-1-yloxytripyrrolidinophosphonium hexafluorophosphate (60 mg, 0.12 mmol) were dissolved in dehydrated DMF (5 mL) and stirred for 14 hours at room temperature. The reaction was stopped by 2N HCl aq. This mixture was extracted by dichloromethane and washed by brine. The organic layer was dried by Na₂SO₄ and the solvent was distilled off under reduced pressure. Some of the byproducts were removed by subjecting the residue to HPLC (eluent, from 20% acetonitrile/0.1% trifluoroacetic acid/water (0 min) to 80% acetonitrile/0.1% trifluoroacetic acid/water (15 min); flow rate=25.0 mL/min).

Trifluoroacetic acid (4 mL) and triethylsilane (100 μL) were added to the crudely purified compound and stirred for four hours at room temperature. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=25.0 mL/min), and 5-aminocyclohexylamide-fluorescein (compound 6, 12.4 mg, 0.026 mmol, yield 27%) was obtained.

¹H NMR (400 μMHz, CD₃OD): δ 1.51-1.63 (m, 4H), 2.13-2.18 (m, 4H), 3.10-3.19 (m, 1H), 3.89-3.96 (m, 1H), 6.59 (dd, 2H, J=2.4, 8.8 Hz), 6.66 (d, 1H, J=8.8 Hz), 6.75 (d, 1H, J=2.4 Hz), 7.33 (d, 1H, J=8.3 Hz), 8.20 (dd, 1H, J=1.5, 8.3 Hz), 8.45 (d, 1H, J=1.5 Hz) ¹³C NMR (75 MHz, DMSO-d₆): δ 29.2, 29.7, 47.5, 48.6, 102.2, 109.0, 112.6, 123.3, 124.1, 126.4, 129.0, 134.7, 136.3, 151.8, 154.6, 157.9, 159.6, 163.9, 168.1 HRMS (ESI⁺): Calcd for [M+H]⁺ 473.1713, Found 473.1699 (−1.4 mmu).

Compound 8 of the present invention was synthesized next according to the following scheme 2.

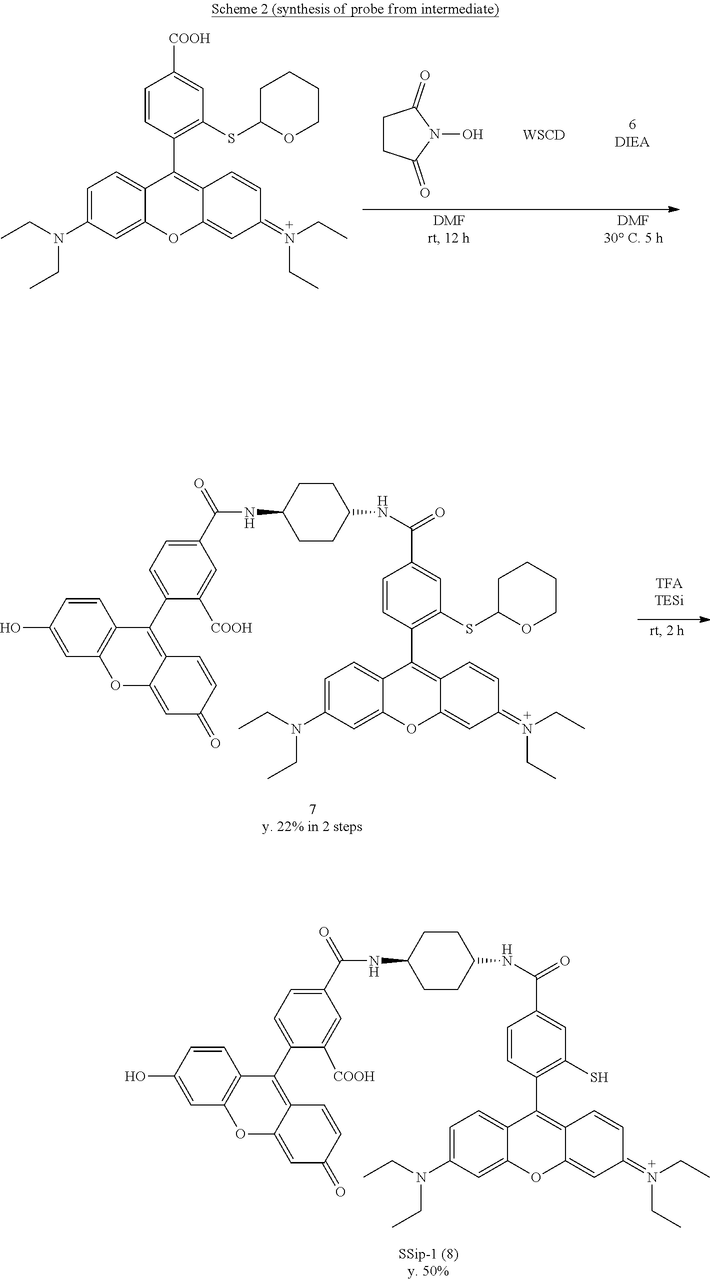

### (7) Synthesis of Compound 7

Compound 5 (10 mg, 0.018 mmol) and N-hydroxysuccinimide (16.1 mg, 0.14 mmol) and 1-(3-dimethylaminopropyl)-3-ethylcarbodiimide hydrochloride (13.5 mg, 0.07 mmol) were dissolved in dehydrated DMF (4 mL) and stirred for 12 hours at room temperature in an argon atmosphere. The reaction was stopped by 0.1N HCl aq. This mixture was extracted by dichloromethane and washed by brine. The organic layer was dried by Na₂SO₄ and the solvent was distilled off under reduced pressure. Some of the byproducts were removed by subjecting the residue to HPLC (eluent, from 20% acetonitrile/0.1% trifluoroacetic acid/water (0 min) to 80% acetonitrile/0.1% trifluoroacetic acid/water (15 min); flow rate=25.0 mL/min).

The crudely purified compound and compound 6 (14 mg, 0.030 mmol) and N,N-diisopropylethylamine (22 mg, 0.17 mmol) were dissolved in dehydrated DMF (5 mL) and stirred for six hours at 30° C. in an argon atmosphere. The reaction was stopped by 0.1N HCl aq. This mixture was extracted by dichloromethane and washed by brine. The organic layer was dried by Na₂SO₄ and the solvent was distilled off under reduced pressure. The residue was purified by HPLC (eluent, from 20% acetonitrile/0.1% trifluoroacetic acid/water (0 min) to 80% acetonitrile/0.1% trifluoroacetic acid/water (15 min); flow rate=5.0 mL/min), and S-pyran SSip-1 (compound 7, 4.0 mg, 0.0039 mmol, yield 22%) was obtained.

¹H NMR (400 μMHz, CD₃OD): δ 1.31 (t, 12H, J=6.4 Hz), 1.45-1.58 (m, 5H), 1.65-1.69 (m, 4H), 1.83-1.84 (m, 1H), 2.16-2.18 (m, 4H), 3.42-3.43 (m, 1H), 3.69 (q, 8H, J=4.0 Hz), 3.76-3.78 (m, 1H), 4.00-4.07 (m, 2H), 5.32-5.34 (m, 1H), 6.52-6.54 (m, 2H), 6.62 (d, 2H, J=8.8 Hz), 6.71 (d, 2H, J=2.4 Hz), 6.98 (d, 2H, J=2.0 Hz), 7.03-7.06 (m, 2H), 7.14-7.16 (m, 2H), 7.30-7.40 (m, 2H), 7.94 (dd, 1H, J=8.1, 1.7 Hz), 8.23 (dd, 1H, J=7.8, 1.5 Hz). ¹³C NMR (100 μMHz, CD₃OD); δ 12.8, 22.1, 26.4, 32.3, 32.5, 46.9, 50.1, 50.2, 65.2, 86.7, 97.4, 103.7, 111.1, 113.8, 114.8, 115.7, 125.1, 125.8, 126.9, 128.7, 130.2, 131.1, 132.6, 132.9, 135.5, 137.4, 137.8, 138.2, 138.4, 154.2, 156.9, 157.3, 157.4, 159.4, 159.5, 167.9, 168.3, 170.5. HRMS (ESI⁺): Calcd for [M]+: 1013.4159, Found 1013.4137 (−2.2 mmu).

### (8) Synthesis of Compound 8

Trifluoroacetic acid (2 mL) and triethylsilane (20 μL) were added to S-pyran SSip-1 (compound 7, 4.0 mg, 0.0039 mmol) and stirred for two hours at room temperature. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=5.0 mL/min), and SSip-1 (1.8 mg, 0.0019 mmol, yield 50%) was obtained. ¹H NMR (400 μMHz, CD₃OD): δ 1.33 (t, 12H, J=7.1 Hz), 1.64-1.68 (m, 4H), 2.13-2.19 (m, 4H), 3.71 (q, 8H, J=7.0 Hz), 4.00-4.06 (m, 2H), 6.96-7.05 (m, 4H), 7.10 (dd, 2H, J=2.4, 9.6 Hz), 7.17-7.30 (m, 6H), 7.42 (d, 2H, J=8.3 Hz), 7.53 (d, 2H, J=7.8 Hz), 7.86 (dd, 1H, J=1.7, 8.1 Hz), 8.11 (d, 1H, J=1.5 Hz), 8.31 (dd, 1H, J=1.5, 7.8 Hz), 8.73 (m, 1H) HRMS (ESI⁺): Calcd for [M]⁺ 929.3584, Found 929.3554 (−3.0 mmu).

A single peak was found at 17 minutes in the HPLC chromatogram after purification (linear gradient from 20% acetonitrile/0.1% trifluoroacetic acid/water to 80% acetonitrile/0.1% trifluoroacetic acid/water (flow rate=1.0 mL/min), Abs. 300 nm).

### Example 1

Na₂S₄ was added to compound 8 synthesized as described above, and the absorption spectrum (UV-1650PC, Shimadzu) and fluorescence spectrum (F-4500, Hitachi) were measured.

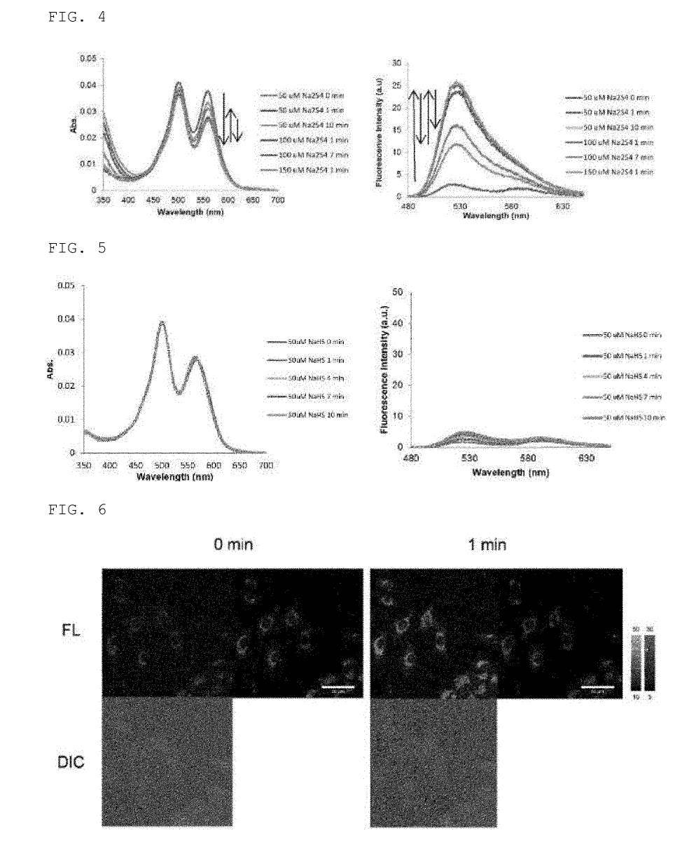

The left drawing of FIG. 3 shows the measurement results of the absorption spectrum of 1 μM of SSip-1 in 0.1 M sodium phosphate buffer (pH 7.4) in the presence of 300 μM of GSH after addition of 50 μM of Na₂S₄ (containing 0.1% DMSO and 1 mg/mL of BSA as co-solvents). The right drawing of FIG. 3 shows the measurement results of the fluorescence spectrum under the same conditions. The excitation wavelength was 470 nm.

A decrease in absorbance on the rhodamine side and a rise in fluorescence derived from fluorescein were seen after addition of 50 μM of Na₂S₄, and SSip-1 served as an OFF/ON type of fluorescent probe. Given that the concentration of glutathione persulfide (GSSH) which is a molecule that includes sulfane sulfur in cells is reported to be 10-100 μM and an approximately 10-fold rise in fluorescence intensity occurred in SSip-1 by addition of 50 μM of Na₂S₄, the probe can be said to be capable of adequately detecting sulfane sulfur in cells.

Reversibility of SSip-1 (In Vitro Assay in the Presence of 5 mM of GSH)

The existence of a response to sulfane sulfur was studied in the presence of 5 mM of GSH, which is the GSH concentration in cells (FIG. 4).

The left drawing of FIG. 4 shows the measurement results of the absorption spectrum of 1 μM of SSip-1 in 0.1 M sodium phosphate buffer (pH 7.4) in the presence of 5 mM of GSH after addition of 50 μM of Na₂S₄ (containing 0.1% DMSO and 1 mg/mL of BSA as co-solvents). The right drawing of FIG. 4 shows the measurement results of the fluorescence spectrum under the same conditions. The excitation wavelength was 470 nm.

As a result, the fluorescence rose after addition of Na₂S₄ in the presence of 5 mM of GSH, and the fluorescence intensity decreased over time. When Na₂S₄ was again added, the fluorescence intensity again rose, showing reversibility.

Selectivity with H₂S

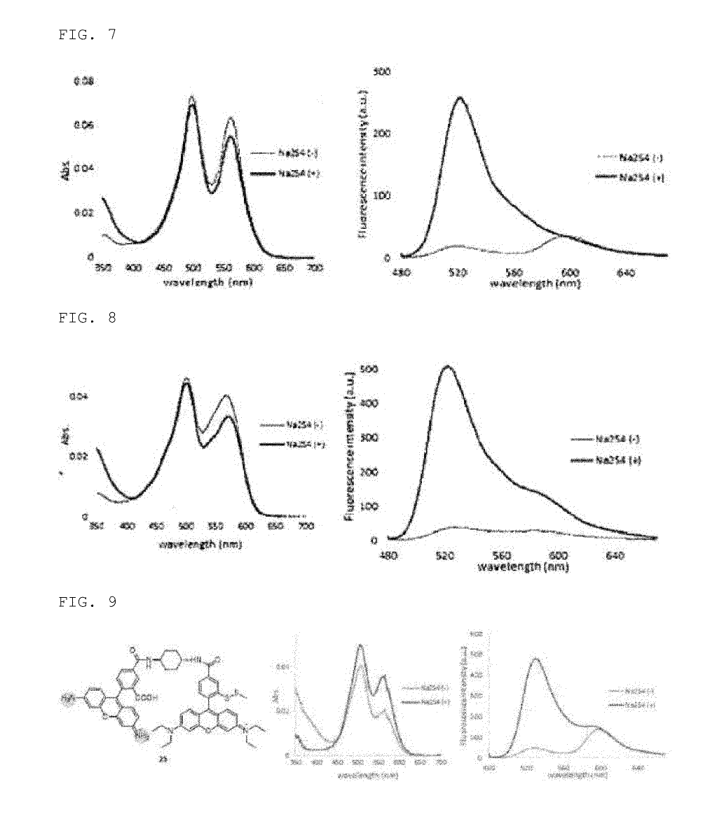

FIG. 5 shows the results obtained by studying the selectivity of SSip-1 with H₂S. The left drawing of FIG. 5 shows the measurement results of the absorption spectrum of 0.1 μm of SSip-1 in 0.1 M sodium phosphate buffer (pH 7.4) in the presence of 300 μM of GSH after addition of 50 μM of NaHS (containing 0.1% DMSO and 1 mg/mL of BSA as co-solvents). The right drawing of FIG. 3 shows the measurement results of the fluorescence spectrum under the same conditions. The excitation wavelength was 470 nm.

As is understood from FIG. 5, virtually no rise in fluorescence intensity was seen even with addition of 50 μM of NaHS, which is an H₂S donor, showing selectivity.

Live Cell Imaging

Given that SSip-1 demonstrated an excellent response to sulfane sulfur in vitro, SSip-1 cell imaging was conducted. SSip-1 was thought to possibly not have cell membrane permeability due to its fluorescein structure. Therefore, a diacetate (DA) form used to make fluorescein cell membrane permeable was applied to SSip-1, and SSip-1 DA was synthesized according to the following scheme.

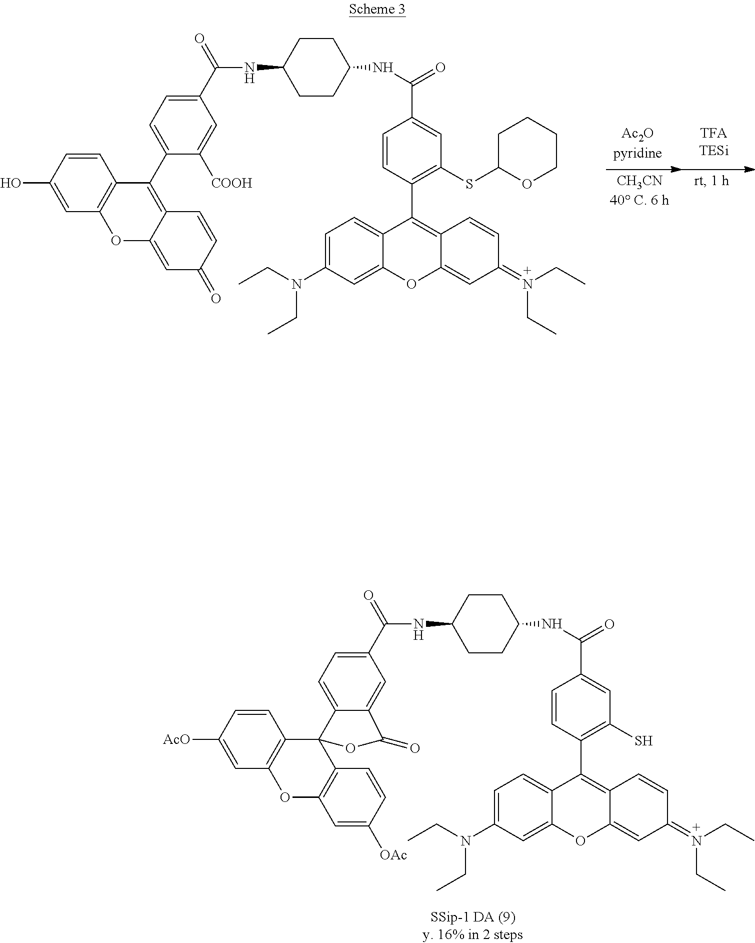

### Synthesis of Compound 9 (SSip-1 DA)

S-pyran SSip-1 (compound 7, 10 mg, 0.0099 mmol) was dissolved in dehydrated acetonitrile (5 mL), and acetic anhydride (1 mL) and pyridine (80 mg, 1.01 mmol) were added and stirred for six hours at 40° C. in an argon atmosphere. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=5.0 mL/min), and some of the byproducts were removed.

The crudely purified compound was dissolved in trifluoroacetic acid (1 mL) and triethylsilane (10 μL) and stirred for one hour at room temperature. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=5.0 mL/min), and SSip-1 DA (1. mg, 0.0018 mmol, yield 18%) was obtained.

¹H NMR (400 μMHz, CD₃OD): δ 1.32 (t, 12H, J=6.8 Hz), 1.60-1.63 (m, 4H), 2.13-2.15 (m, 4H), 2.30 (s, 6H), 3.71 (q, 8H, J=6.7 Hz), 3.94-4.02 (m, 2H), 6.85-6.94 (m, 4H), 7.01 (d, 2H, J=2.4 Hz), 7.10 (dd, 2H, J=2.2, 9.5 Hz), 7.17-7.18 (m, 2H), 7.20-7.21 (m, 2H), 7.34-7.42 (m, 2H), 7.85 (dd, 1H, J=7.8, 2.0 Hz), 8.09 (m, 1H), 8.22-8.24 (m, 1H), 8.47-8.48 (m, 1H) HRMS (ESI⁺): Calcd for [M]⁺ 1013.3795, Found 1013.3764 (−3.1 mmu).

A single peak was found at 8 minutes in the HPLC chromatogram after purification (linear gradient from 80% acetonitrile/0.1% trifluoroacetic acid/water to 20% acetonitrile/0.1% trifluoroacetic acid/water (flow rate=1.0 mL/min), Abs. 550 nm).

The SSip-1 DA was introduced into A549 cells, and 500 μM of Na₂S₄ was added outside the cells under a microscope. The A549 cells were incubated for three hours by 10 μM of SSip-1 DA (containing 0.0003% Purulonic and 1% DMSO as a co-solvent). The excitation wavelength was 488 nm, and the emission wavelengths were 500-570 nm and 590-650 nm.

As a result, the fluorescence intensity rose, and SSip-1 detected sulfane sulfur within the cells (FIG. 6). Furthermore, Purulonic, which is a surfactant, was used when introducing the probe, and aggregation of the probe was averted. In addition, when the fluorescence intensity of the two wavelengths of the fluorescein-derived fluorescence wavelength region (500-570 nm) and the rhodamine-derived fluorescence wavelength region (590-650 nm) was calculated, the intensity of the fluorescein-derived fluorescence wavelength region increased, and the intensity of the rhodamine-derived fluorescence wavelength region decreased slightly. Therefore, SSip-1 was thought to permit not only imaging based on changes in the fluorescence intensity from fluorescein, but also ratio measurement in cell imaging under this measurement.

### Synthesis Example 2

### Synthesis of Compound a1 (6-Fluorescein-SSip-1) of the Present Invention

Compound a1 of the present invention was synthesized according to the following scheme 4.

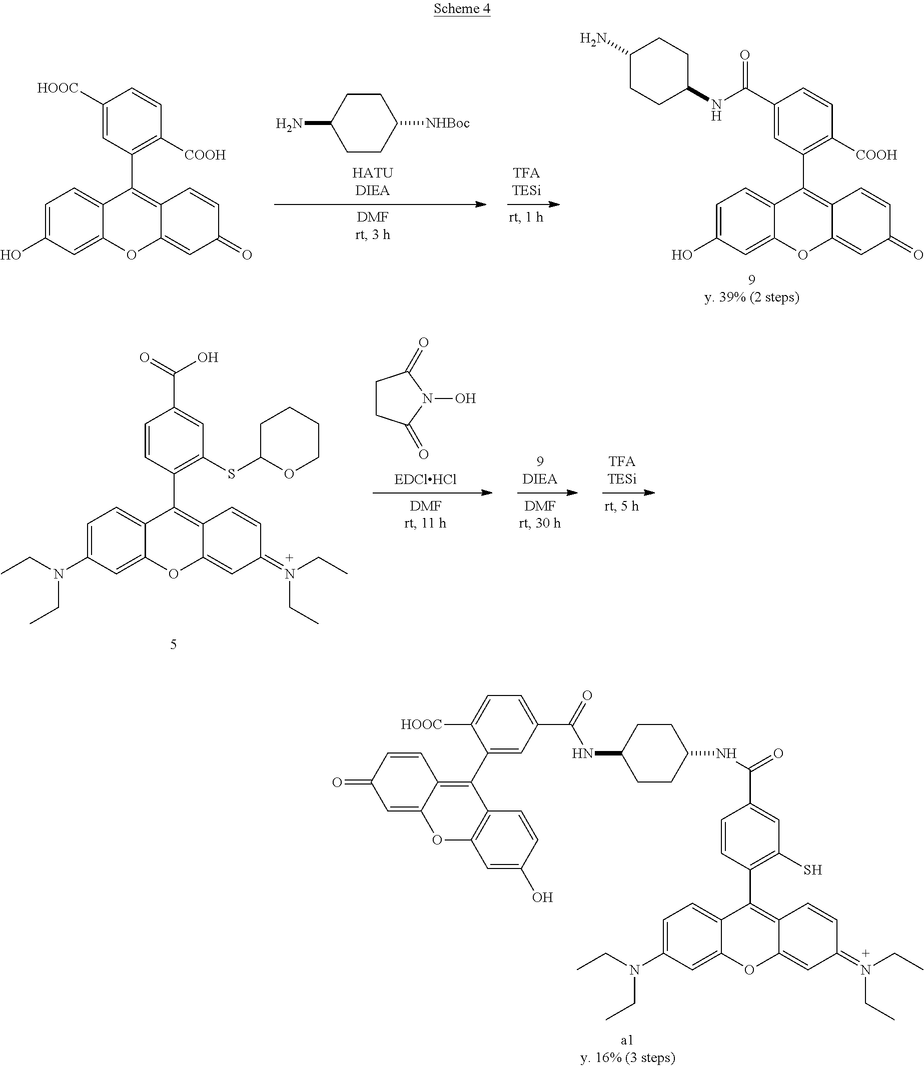

### (1) Synthesis of Compound 9

6-Carboxyfluorescein (200 mg, 0.53 mmol) and N-Boc-trans-1,4-cyclohexanediamine (285 mg, 1.33 mmol) and N,N-diisopropylethylamine (686 mg, 5.32 mmol) and O-(7-azabenzotriazol-1-yl)-N,N,N′,N′-tetramethyluronium hexafluorophosphate (HATU) (403 mg, 1.06 mmol) were dissolved in dehydrated DMF (5 mL) and stirred for three hours at room temperature. The reaction was stopped by 1N HCl aq. This mixture was extracted by dichloromethane, and the organic layer was washed by brine, and the solvent was distilled off under reduced pressure after drying by Na₂SO₄. Some of the byproducts and reagent residues were then removed by subjecting the residue to HPLC (eluent, from 20% acetonitrile/0.1% trifluoroacetic acid/water (0 min) to 80% acetonitrile/0.1% trifluoroacetic acid/water (15 min); flow rate=5.0 mL/min).

Trifluoroacetic acid (4 mL) and triethylsilane (100 μL) were added to the crudely purified compound and stirred for one hour at room temperature. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=25.0 mL/min), and 6-aminocyclohexylamide-fluorescein (compound 9, 98.3 mg, 0.21 mmol, yield 39%) was obtained.

¹H NMR (300 μMHz, CD₃OD): δ 1.45-1.52 (4H, m), 2.00-2.07 (4H, m), 3.07 (1H, br m), 3.82 (1H, br m), 6.62 (2H, dd, J=8.8, 2.2 Hz), 6.71 (2H, d, J=8.8 Hz), 6.78 (2H, d, J=2.2 Hz), 7.64 (1H, d, J=1.5 Hz), 8.11 (1H, d, J=8.1 Hz), 8.15 (1H, dd, J=8.1, 1.5 Hz) ¹³C NMR (75 μMHz, DMSO-d₆): δ 29.14, 29.61, 47.59, 48.60, 102.26, 109.13, 112.77, 122.16, 124.78, 128.25, 129.23, 129.58, 140.65, 151.82, 152.56, 157.32, 159.65, 163.76, 168.00. HRMS (ESI⁺): calcd for [M+H]⁺ , 473.1713, found, 473.1680 (−3.3 mmu).

### (2) Synthesis of Compound a1

Compound 5 (20.0 mg, 0.036 mmol) and N-hydroxysuccinimide (37.0 mg, 0.32 mmol) and 1-(3-dimethylaminopropyl)-3-ethylcarbodiimide hydrochloride (31.0 mg, 0.16 mmol) were dissolved in dehydrated DMF (4 mL) and stirred for 11 hours at room temperature. After distilling off the solvent under reduced pressure, the residue was crudely purified by HPLC (eluent, from 20% acetonitrile/0.1% trifluoroacetic acid/water (0 min) to 80% acetonitrile/0.1% trifluoroacetic acid/water (15 min); flow rate=5.0 mL/min).

The crudely purified compound and compound 9 (14 mg, 0.030 mmol) and N,N-diisopropylethylamine (22 mg, 0.17 mmol) were dissolved in dehydrated DMF (5 mL) and stirred for 30 hours at room temperature. The reaction was stopped by 0.1N HCl aq. This mixture was extracted by dichloromethane, and the organic layer was washed by brine, and the solvent was distilled off under reduced pressure after drying by Na₂SO₄. Trifluoroacetic acid (1 mL) and triethylsilane (10 μL) were added to the residue and stirred for three hours at room temperature. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=5.0 mL/min), and compound a1 (4.3 mg, 4.62 μmol, yield in three steps 61%) was obtained.

¹H NMR (400 μMHz, CD₃OD): δ 1.30 (12H, t, J=7.1 Hz), 1.50-1.52 (4H, m), 2.02-2.03 (4H, m), 3.68 (8H, q, J=7.3 Hz), 3.88 (2H, br), 6.53-6.56 (4H, m), 6.60-6.62 (2H, m), 6.69-6.70 (2H, m), 6.88 (2H, d, J=9.2 Hz), 6.93-6.97 (4H, m), 7.43 (1H, d, J=7.8 Hz), 7.63 (1H, br), 7.99 (1H, dd, J=8.1, 1.7 Hz), 8.06 (1H, d, J=7.8 Hz), 8.12-8.14 (1H, m), 8.22 (1H, d, J=1.5 Hz) HRMS (ESI⁺): calcd for [M]²⁺, 1857.7045, found, 1857.7021 (−2.4 mmu).

The HPLC chromatogram after purification was as follows. The solution was done with a liner gradient from 20% CH₃CN/0.1% trifluoroacetic acid aq. (0 min) to 80% CH₃CN/0.1% trifluoroacetic acid aq. (15 min) (flow rate=1.0 mL/min).

### Synthesis Example 3

### Synthesis of Compound a2 (5-Amide-SSip-1) of the Present Invention

Compound a2 of the present invention was synthesized according to the following scheme 5.

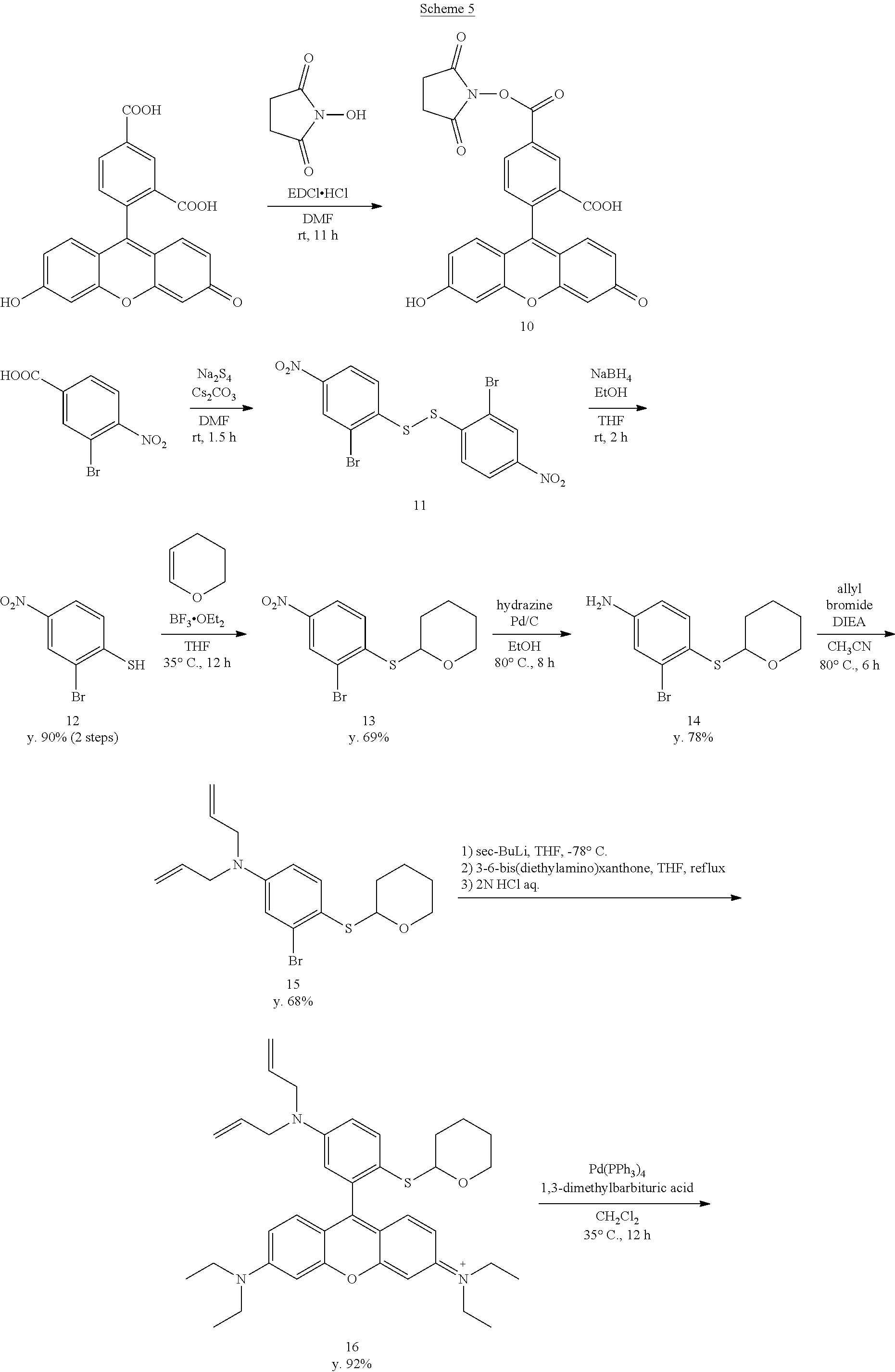

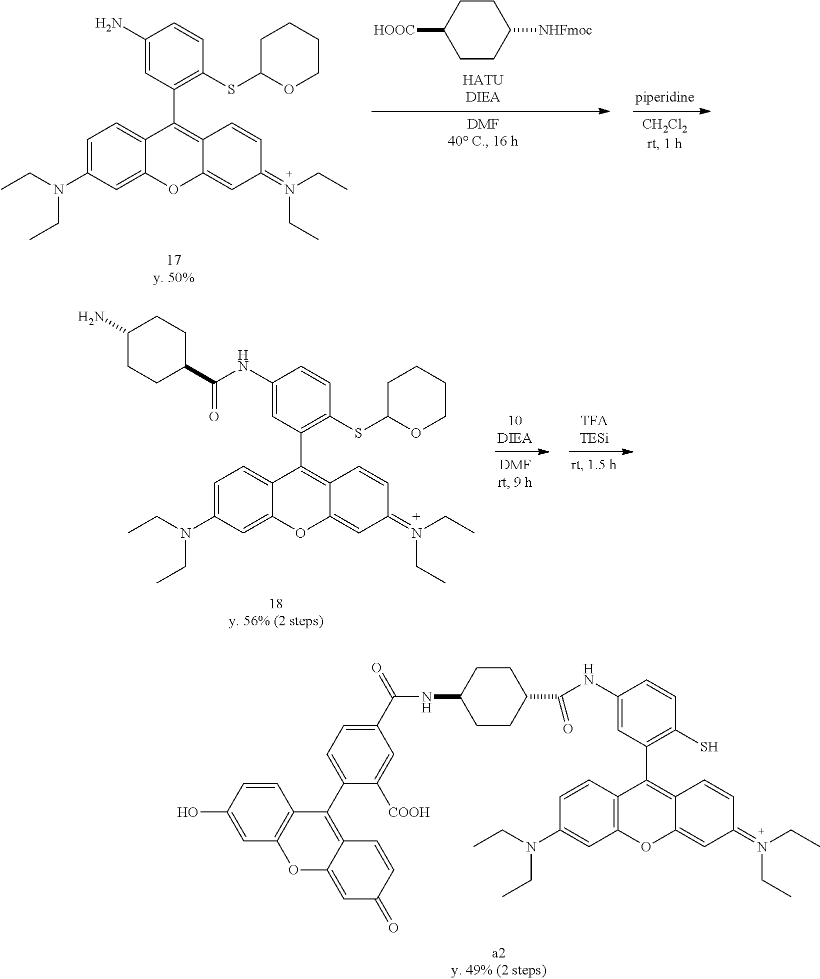

### (1) Synthesis of Compound 12

Sodium tetrasulfide (Na₂S₄, 4.37 g, 25.1 mmol) was dissolved in dehydrated DMF, and 2-bromo-1-fluoro-4-nitrobenzene (5.0 g, 22.8 mmol) was added and stirred for 1.5 hour at room temperature. The reaction solution was cooled to 0° C., and the reaction was stopped by adding 2N HCl aq. After adding dichloromethane, this mixture was filtered by a Kiriyama funnel, and the insoluble matter was removed. The filtrate was extracted by dichloromethane, and the organic layer was washed by water and brine, and the solvents other than DMF were distilled off under reduced pressure after drying by Na₂SO₄. The residue was filtered by a Kiriyama funnel, and the solid that did not dissolve in DMF was removed. The filtrate was distilled off under reduced pressure, and the residue was washed by dichloromethane, and crudely purified compound 11 was obtained. Compound 11 was dissolved in a mixed solvent of THF (60 mL) and ethanol (20 mL) and cooled to 0° C. Sodium borohydride (489 mg, 12.9 mmol) was added and stirred for two hours at room temperature. The reaction solution was cooled to 0° C., and the reaction was stopped by adding 2N HCl aq. After distilling off the organic solvent under reduced pressure, the precipitated solid was filtered out by a Kiriyama funnel, washed by water, and 2-bromo-4-nitrobenzenethiol (compound 12, 4.8 g, 20.5 mmol, yield in two steps 90%) was obtained.

¹H NMR (300 μMHz, CDCl₃): δ 4.37 (1H, s), 7.49 (1H, d, J=8.8 Hz), 8.04 (1H, dd, J=8.4, 2.6 Hz), 8.42 (1H, d, J=2.2 Hz) ¹³C NMR (75 μMHz, CDCl₃): δ 121.16, 122.57, 127.99, 128.71, 144.51. HRMS (ESI⁻): calcd for [MH]⁻, 231.9068, found, 231.9064 (−0.4 mmu).

### (2) Synthesis of Compound 13

Compound 12 (1.36 g, 5.81 mmol) was dissolved in dehydrated THF (30 mL), and 3,4-dihydro-2H-pyran (4.88 g, 58.1 mmol) was added, and the solution was cooled to 0° C. in an argon atmosphere. A boron trifluoride-ethyl ether complex (826 mg, 5.81 mmol) was added thereto and stirred for 12 hours at 35° C. After returning the reaction solution to room temperature, the reaction was stopped by adding saturated sodium bicarbonate aqueous solution. This mixture was extracted by ethyl acetate, the organic layer was washed by brine, dried by Na₂SO₄, and the solvent was distilled off under reduced pressure. The residue was purified by column chromatography (silica gel, ethyl acetate/n-hexane), and 2-((2-bromo-4-nitrophenyl)thio)tetrahydro-2H-pyran (compound 13, 1.28 g, 4.03 mmol, yield 69%) was obtained.

¹H NMR (300 μMHz, CDCl₃): δ 1.67-1.82 (3H, m), 1.92-1.97 (2H, m), 2.14-2.18 (1H, m), 3.66-3.73 (1H, m), 4.09-4.13 (1H, m), 5.53 (1H, m), 7.70 (1H, d, J=8.8 Hz), 8.11 (1H, dd, J=8.8, 2.9 Hz), 8.37 (1H, d, J=2.9 Hz)

¹³C NMR (75 μMHz, CDCl₃): 520.84, 25.27, 30.96, 63.90, 82.98, 121.32, 122.51, 127.14, 127.26, 127.46, 145.09, 148.10

### (3) Synthesis of Compound 14

Compound 13 (1.28 g, 4.03 mmol) was dissolved in ethanol (40 mL) in an argon atmosphere, and palladium 10% on carbon (128 mg) was added. Hydrazine hydrate (50% in H₂O, 644 mg, 20.1 mmol) dissolved in ethanol (10 mL) was added dropwise thereto and stirred for eight hours at 80° C. After cooling to room temperature, the reaction solution was filtered by Celite, and the palladium was removed. After distilling off the filtrate under reduced pressure, the residue was purified by column chromatography (silica gel, n-hexane/dichloromethane), and 3-bromo-4-((tetrahydro-2H-pyran-2-yl)thio)aniline (compound 14, 0.90 g, 3.13 mmol, yield 78%) was obtained.

¹H NMR (300 μMHz, CDCl₃): δ 1.57-1.67 (3H, m), 1.83-1.88 (2H, m), 2.01-2.04 (1H, m), 3.52-3.59 (1H, m), 3.75 (2H, s), 4.14-4.22 (1H, m), 5.13 (1H, m), 6.56 (1H, dd, J=8.4, 2.6 Hz), 6.93 (1H, d, J=2.9 Hz), 7.40 (1H, d, J=8.1 Hz)

¹³C NMR (75 μMHz, CDCl₃): δ 21.27, 25.55, 31.23, 64.23, 85.62, 114.65, 119.04, 122.91, 128.77, 135.65, 147.17

HRMS (ESI⁺): calcd for [M+H]⁺ , 288.0058, found, 288.0049 (−0.9 mmu).

### (4) Synthesis of Compound 15

Compound 14 (0.80 mg, 2.78 mmol) was dissolved in dehydrated acetonitrile (15 mL), and N,N-diisopropylethylamine (3.59 g, 27.8 mmol) and allyl bromide (1.68 g, 13.9 mmol) were added and stirred for six hours at 80° C. in an argon atmosphere. After cooling the reaction solution to room temperature, water was added. This mixture was extracted by dichloromethane, and the organic layer was washed by 1N HCl aq. and brine, and the solvent was distilled off under reduced pressure after drying by Na₂SO₄. The residue was purified by column chromatography (silica gel, n-hexane/dichloromethane), and N,N-diallyl-3-bromo-4-((tetrahydro-2H-pyran-2-yl)thio)aniline (compound 15, 0.69 g, 1.87 mmol, yield 68%) was obtained.

¹H NMR (300 MHz, acetone-d₆): δ 1.57-1.63 (3H, m), 1.69-1.85 (2H, m), 1.94-1.99 (1H, m), 3.46-3.53 (1H, m), 3.97-3.98 (4H, m), 4.07-4.14 (1H, m), 5.11-5.17 (4H, m), 5.20 (1H, m), 5.81-5.93 (2H, m), 6.67 (1H, dd, J=8.8, 2.9 Hz), 6.93 (1H, d, J=2.9 Hz), 7.40 (1H, d, J=8.8 Hz)

¹³C NMR (75 μMHz, CDCl₃): δ 21.27, 25.59, 31.25, 52.61, 64.19, 85.82, 111.87, 116.22, 116.40, 120.02, 129.55, 132.90, 135.80, 149.22

HRMS (ESI⁺): calcd for [M+H]⁺ , 368.0684, found, 368.0683 (−0.1 mmu).

### (5) Synthesis of Compound 16

Compound 15 (229 mg, 0.62 mmol) was added to a dried, argon-purged and dissolved in dehydrated tetrahydrofuran (10 mL). After cooling to −78° C., 1 M sec-butyllithium (620 μL, 0.62 mmol) was added and stirred for 20 minutes. 3,6-Bis(diethylamino)xanthone (30 mg, 0.089 mmol) dissolved in dehydrated tetrahydrofuran (5 mL) was slowly added at the same temperature and, after returning to room temperature, stirred for one hour at 70° C. After cooling to room temperature, the reaction was stopped by 2N HCl aq. This mixture was extracted by dichloromethane, and the organic layer was washed by brine and the solvent was distilled off under reduced pressure after drying by Na₂SO₄. The residue was purified by column chromatography (silica gel, dichloromethane/methanol), and 5-N-diallyl-2-S-THP-tetraethylrhodamine (compound 16, 50 mg, 0.082 mmol, yield 92%) was obtained.

¹H NMR (300 MHz, acetone-d₆): δ 1.33 (12H, t, J=7.0 Hz), 1.39-1.54 (5H, m), 1.65-1.69 (1H, m), 3.14-3.21 (1H, m), 3.60-3.67 (1H, m), 3.77 (8H, q, J=7.1 Hz), 4.04 (4H, d, J=5.1 Hz), 4.83 (1H, m), 5.15-5.19 (4H, m), 5.83-5.91 (2H, m), 6.71 (1H, d, J=2.9 Hz), 6.94-6.96 (2H, m), 6.99 (2H, dd, J=8.8, 2.9 Hz), 7.14-7.20 (2H, m), 7.28 (1H, d, J=9.5 Hz), 7.30 (1H, d, J=9.5 Hz), 7.64 (1H, d, J=8.8 Hz)

¹³C NMR (75 μMHz, acetone-d₆): δ 12.77, 21.91, 26.08, 32.28, 46.51, 53.41, 64.46, 87.77, 96.70, 96.81, 114.04, 114.64, 114.72, 114.85, 115.04, 115.11, 116.60, 119.25, 132.96, 133.47, 134.31, 137.54, 137.55, 137.85, 149.05, 156.57, 156.69, 158.70, 158.89, 159.25

HRMS (ESI⁺): calcd for [M]⁺ , 610.3467, found, 610.3449 (−1.8 mmu).

### (6) Synthesis of Compound 17

1,3-dimethylbarbituric acid (74 mg, 0.48 mmol) and tetrakis(triphenylphosphine)palladium(0) (11 mg, 9.5 μmol) were added to an argon-purged flask, and compound 16 (58 mg, 0.095 mmol) dissolved in dehydrated dichloromethane (15 mL) was added and stirred for 12 hours at 35° C. After cooling to room temperature, the reaction solution was washed by saturated sodium carbonate aqueous solution and brine, and the solvent was distilled off under reduced pressure after drying by Na₂SO₄. The residue was purified by column chromatography (silica gel, dichloromethane/methanol), and 5-amino-2-S-THP-tetraethylrhodamine (compound 17, 25 mg, 0.047 mmol, yield 50%) was obtained.

¹H NMR (300 MHz, acetone-d₆): δ 1.33 (12H, t, J=7.0 Hz), 1.45-1.62 (6H, m), 3.15-3.19 (1H, m), 3.59-3.66 (1H, m), 3.77 (8H, q, J=7.1 Hz), 4.76 (1H, m), 5.61 (2H, s), 6.73 (1H, d, J=2.2 Hz), 6.96 (3H, m), 6.99 (3H, m), 7.18 (1H, dd, J=9.5, 2.2 Hz), 7.20 (1H, dd, J=9.5, 2.2 Hz), 7.30 (1H, d, J=9.5 Hz), 7.33 (1H, d, J=9.5 Hz), 7.52 (1H, d, J=8.1 Hz)

¹³C NMR (75 μMHz, acetone-d₆): δ 12.80, 22.02, 26.08, 32.31, 46.50, 64.60, 87.92, 96.72, 96.82, 114.60, 114.67, 114.88, 115.06, 115.54, 117.13, 118.61, 132.98, 133.49, 137.97, 138.14, 150.35, 156.55, 156.67, 158.66, 158.84, 159.38

HRMS (ESI⁺): calcd for [M]⁺ , 530.2841, found, 530.2830 (−1.0 mmu).

### (7) Synthesis of Compound 18

Compound 17 (10 mg, 19 μmol), N-Fmoc-trans-1,4-cyclohexanediamine (21 mg, 57 μmol), N,N-diisopropylethylamine (25 mg, 0.19 mmol), and O-(7-azabenzotriazol-1-yl)-N,N,N′,N′-tetramethyluronium hexafluorophosphate (HATU) (22 mg, 57 μmol) were dissolved in dehydrated DMF (3 mL) and stirred for 16 hours at 40° C. After cooling the reaction solution to room temperature, the reaction was stopped by 1N HCl aq. This mixture was extracted by dichloromethane, and the organic layer was washed by brine, and the solvent was distilled off under reduced pressure after drying by Na₂SO₄. The crudely purified product was dissolved in dichloromethane (5 mL), and piperidine (0.50 mL) was added and stirred for one hour at room temperature. The reaction was stopped by 1N HCl aq. This mixture was extracted by dichloromethane, and the organic layer was washed by brine, and the solvent was distilled off under reduced pressure after drying by Na₂SO₄. The residue was purified by HPLC (eluent, from 20% acetonitrile/0.1% trifluoroacetic acid/water (0 min) to 80% acetonitrile/0.1% trifluoroacetic acid/water (20 min); flow rate=5.0 mL/min), and 5-aminocyclohexylamide-2-S-THP-tetraethylrhodamine (compound 18, 6.0 mg, 11 μmol, yield in two steps 56%) was obtained.

¹H NMR (300 μMHz, CD₃OD): δ 1.31 (12H, t, J=7.0 Hz), 1.48-1.59 (9H, m), 1.76-1.79 (1H, m), 1.94-1.98 (4H, m), 2.34-2.38 (1H, m), 2.72-2.76 (1H, m), 3.55 (1H, m), 3.63 (1H, m), 3.69 (8H, q, J=7.1 Hz), 5.08-5.09 (1H, m), 6.98-6.99 (2H, m), 7.06-7.08 (2H, m), 7.21 (1H, d, J=7.3 Hz), 7.24 (1H, d, J=7.3 Hz), 7.69-7.71 (2H, m), 7.82 (1H, d, J=8.1 Hz).

HRMS (ESI⁺): calcd for [M]⁺ , 571.3107, found, 571.3117 (+1.0 mmu).

The HPLC chromatogram after purification was as follows. The solution was done with a liner gradient from 20% CH₃CN/0.1% trifluoroacetic acid aq. (0 min) to 80% CH₃CN/0.1% trifluoroacetic acid aq. (15 min) (flow rate=1.0 mL/min).

### (8) Synthesis of Compound a2

5-carboxyfluorescein (50.0 mg, 0.13 mmol) and N-hydroxysuccinimide (76.0 mg, 0.66 mmol) and 1-(3-dimethylaminopropyl)-3-ethylcarbodiimide hydrochloride (77.0 mg, 0.40 mmol) were dissolved in dehydrated DMF (5 mL) and stirred for one hour at room temperature. After distilling off the solvent under reduced pressure, the residue was subjected to HPLC (eluent, from 20% acetonitrile/0.1% trifluoroacetic acid/water (0 min) to 80% acetonitrile/0.1% trifluoroacetic acid/water (15 min); flow rate=5.0 mL/min), and a crudely purified compound 10 was obtained. Compound 18 (3.0 mg, 4.6 μmol), compound 10 (6.5 mg, 14 μmol), and N,N-diisopropylethylamine (5.9 mg, 46 μmol) were dissolved in dehydrated DMF (5 mL) and stirred for nine hours at room temperature. After distilling off the solvent under reduced pressure, trifluoroacetic acid (TFA, 0.50 mL) and triethylsilane (10 μL) were added to the residue and stirred for 1.5 hour at room temperature. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=5.0 mL/min), and compound a2 (2.1 mg, 2.2 μmol, yield in two steps 49%) was obtained.

¹H NMR (400 μMHz, CD₃OD): δ 1.30 (12H, t, J=6.8 Hz), 1.50-1.56 (2H, m), 1.71-1.74 (2H, m), 2.03-2.04 (2H, m), 2.15-2.16 (2H, m), 2.45 (1H, br m), 3.68 (8H, q, J=7.0 Hz), 3.94 (1H, br m), 6.53 (2H, dd, J=8.8, 2.4 Hz), 6.58 (2H, d, J=8.8 Hz), 6.69 (2H, d, J=2.4 Hz), 6.88 (2H, dd, J=9.3, 2.0 Hz), 6.93 (2H, d, J=9.7 Hz), 6.98 (1H, d, J=2.0 Hz), 7.29 (1H, d, J=7.8 Hz), 7.66 (1H, d, J=2.0 Hz), 7.72 (1H, d, J=8.2 Hz), 7.83 (1H, dd, J=8.8, 2.4 Hz), 8.19 (1H, dd, J=8.1, 1.2 Hz), 8.41 (1H, d, J=1.5 Hz)

HRMS (ESI⁺): calcd for [M]²⁺, 1857.7045, found, 1857.6998 (−4.7 mmu).

The HPLC chromatogram after purification was as follows. The solution was done with a liner gradient from 20% CH₃CN/0.1% trifluoroacetic acid aq. (0 min) to 80% CH₃CN/0.1% trifluoroacetic acid aq. (15 min) (flow rate=1.0 mL/min).

### Synthesis Example 4

### Synthesis of Compound a3 (S-Methyldisulfanyl-SSip-1 DA) of the Present Invention

Compound a3 of the present invention was synthesized according to the following scheme 6.

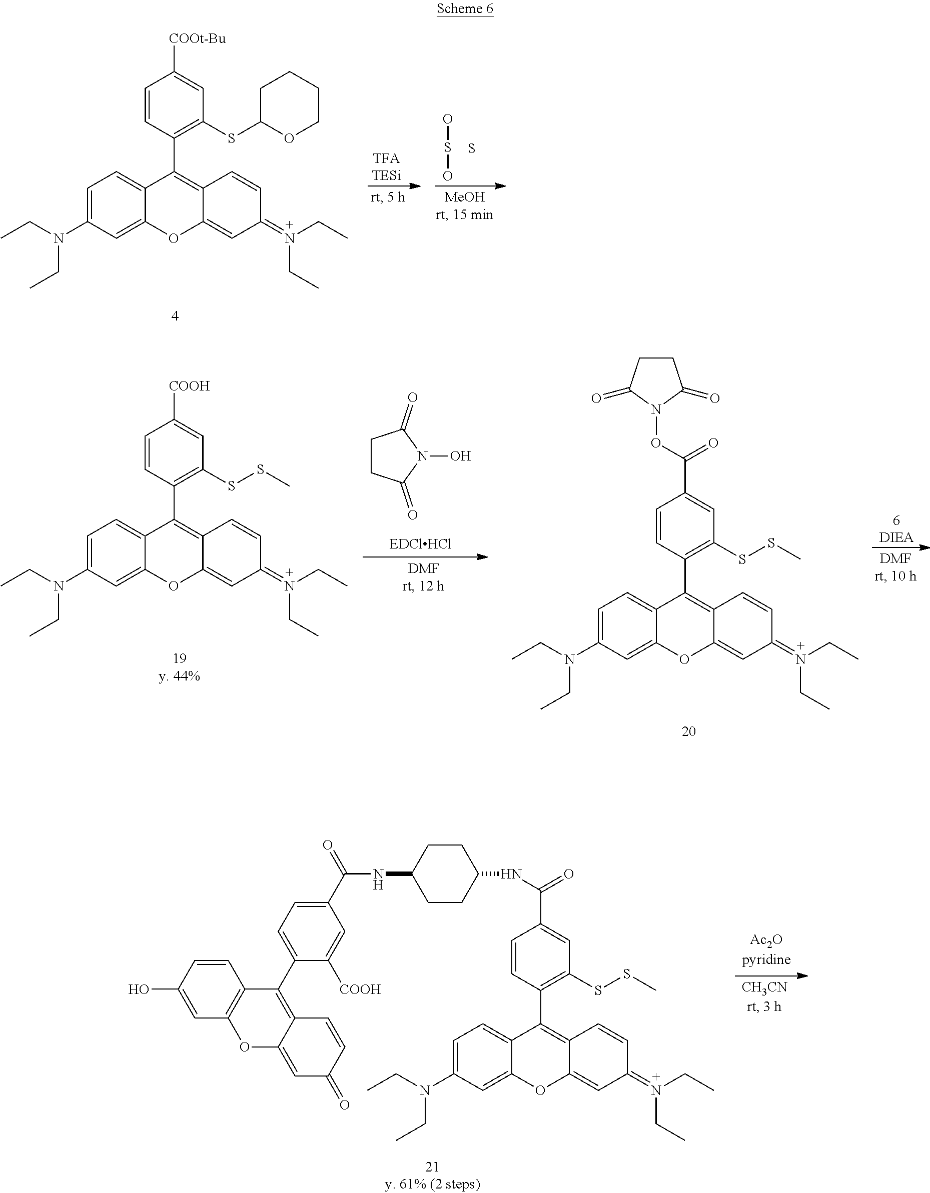

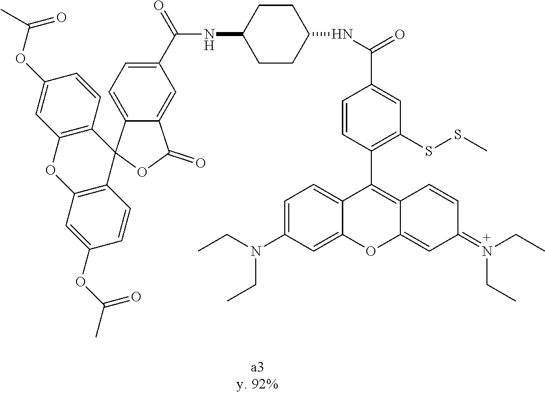

### (1) Synthesis of Compound 19

Trifluoroacetic acid (0.50 mL) and triethylsilane (10 μL) were added to compound 4 (12.9 mg, 20.9 μmol) and stirred for five hours at room temperature. After distilling off the solvent under reduced pressure, the residue was crudely purified by HPLC (eluent, from 20% acetonitrile/0.1% trifluoroacetic acid/water (0 min) to 80% acetonitrile/0.1% trifluoroacetic acid/water (15 min); flow rate=5.0 mL/min). Methyl methane thiosulfate (7.9 mg, 63 μmol) was dissolved in methanol (3 mL), and the crudely purified compound dissolved in methanol (2 mL) was added in small amounts thereto and stirred for 15 minutes at room temperature. After distilling off the solvent under reduced pressure, the residue was purified by HPLC (eluent, from 20% acetonitrile/0.1% trifluoroacetic acid/water (0 min) to 80% acetonitrile/0.1% trifluoroacetic acid/water (15 min); flow rate=5.0 mL/min), and 2-methyldisulfanyl-4-carboxytetraeethylrhodamine (compound 19, 4.8 mg, 9.21 μmol, yield 44%) was obtained.

¹H NMR (300 μMHz, CD₃OD): δ 1.32 (12H, t, J=7.0 Hz), 2.38 (3H, s), 3.70 (8H, q, J=7.1 Hz), 7.01 (2H, d, J=2.2 Hz), 7.09 (2H, dd, J=9.5, 2.2 Hz), 7.15 (2H, d, J=9.5 Hz), 7.48 (1H, d, J=8.1 Hz), 8.16 (1H, dd, J=8.1, 1.5 Hz), 8.71 (1H, d, J=1.5 Hz)

¹³C NMR (100 μMHz, CD₃OD): 512.80, 23.30, 46.98, 97.61, 114.43, 115.94, 129.40, 130.13, 131.61, 132.42, 134.84, 136.95, 138.71, 155.12, 157.51, 159.38, 168.33

HRMS (ESI⁺): calcd for [M]⁺ , 521.1933, found, 521.1895 (−3.8 mmu).

### (2) Synthesis of Compound 21

Compound 19 (4.8 mg, 9.21 μmol) and N-hydroxysuccinimide (9.5 mg, 0.083 mmol) and 1-(3-dimethylaminopropyl)-3-ethylcarbodiimide hydrochloride (8.0 mg, 0.041 mmol) were dissolved in dehydrated DMF (4 mL) and stirred for 12 hours at room temperature. After distilling off the solvent under reduced pressure, crudely purified compound 20 was obtained from the residue by HPLC (eluent, from 20% acetonitrile/0.1% trifluoroacetic acid/water (0 min) to 80% acetonitrile/0.1% trifluoroacetic acid/water (15 min); flow rate=5.0 mL/min).

The crudely purified compound 20, compound 6 (13.1 mg, 0.028 mmol), and N,N-diisopropylethylamine (12 mg, 0.092 mmol) were dissolved in dehydrated DMF (5 mL) and stirred for 10 hours at room temperature. The reaction was then stopped by 0.1N HCl aq. This mixture was extracted by dichloromethane, and the organic layer was washed by brine and the solvent was distilled off under reduced pressure after drying by Na₂SO₄. The residue was refined by HPLC (eluent, from 20% acetonitrile/0.1% trifluoroacetic acid/water (0 min) to 80% acetonitrile/0.1% trifluoroacetic acid/water (15 min); flow rate=5.0 mL/min), and S-methyldisulfanyl-SSip-1 (compound 21, 4.3 mg, 4.62 μmol, yield in two steps 61%) was obtained.

¹H NMR (400 μMHz, CD₃OD): δ 1.32 (12H, t, J=6.8 Hz), 1.70-1.72 (4H, m), 2.18-2.20 (4H, m), 2.37 (3H, s), 3.69 (8H, q, J=7.0 Hz), 4.06-4.08 (2H, br), 6.62 (2H, dd, J=8.8, 2.0 Hz), 6.72 (2H, d, J=8.8 Hz), 6.79 (2H, d, J=2.0 Hz), 6.99 (2H, d, J=2.0 Hz), 7.05 (2H, dd, J=9.8, 2.4 Hz), 7.14 (2H, d, J=9.8 Hz), 7.33 (1H, d, J=7.8 Hz), 7.98 (1H, dd, J=7.8, 1.5 Hz), 8.24 (1H, dd, J=7.8, 1.5 Hz), 8.52-8.53 (2H, m)

HRMS (ESI⁺): calcd for [M]⁺ , 975.3461, found, 975.3444 (−1.7 mmu).

The HPLC chromatogram after purification was as follows. The solution was done with a liner gradient from 20% CH₃CN/0.1% trifluoroacetic acid aq. (0 min) to 80% CH₃CN/0.1% trifluoroacetic acid aq. (15 min) (flow rate=1.0 mL/min).

### (3) Synthesis of Compound a3

Compound 21 (1.8 mg, 1.85 μmol) was dissolved in dehydrated acetonitrile (5 mL), and acetic anhydride (15 μL) and pyridine (29 mg, 0.037 mmol) were added and stirred for three hours at room temperature. After distilling off the solvent under reduced pressure, the residue was refined by HPLC (eluent, from 20% acetonitrile/0.1% trifluoroacetic acid/water (0 min) to 80% acetonitrile/0.1% trifluoroacetic acid/water (15 min); flow rate=5.0 mL/min), and compound a3 (1.8 mg, 170 μmol, yield 92%) was obtained.

¹H NMR (300 μMHz, CD₃OD): δ 1.32 (12H, t, J=7.0 Hz), 1.63-1.67 (4H, m), 2.15-2.17 (4H, m), 2.30 (6H, s), 2.38 (3H, s), 3.71 (8H, q, J=6.8 Hz), 4.03 (2H, br), 6.91-6.93 (4H, m), 7.01 (2H, d, J=2.2 Hz), 7.09 (2H, dd, J=9.5, 2.2 Hz), 7.16 (2H, d, J=9.5 Hz), 7.21 (2H, d, J=2.2 Hz), 7.38 (1H, d, J=8.1 Hz), 7.46 (1H, d, J=8.1 Hz), 7.97 (1H, dd, J=8.1, 1.5 Hz), 8.24 (1H, dd, J=8.1, 1.5 Hz), 8.49 (1H, d, J=1.5 Hz), 8.53 (1H, d, J=1.5 Hz)

HRMS (ESI⁺): calcd for [M]⁺ , 1059.3673, found, 1059.3629 (−4.4 mmu).

The HPLC chromatogram after purification was as follows. The solution was done with a liner gradient from 20% CH₃CN/0.1% trifluoroacetic acid aq. (0 min) to 80% CH₃CN/0.1% trifluoroacetic acid aq. (15 min) (flow rate=1.0 mL/min).

### Synthesis Example 5

### Synthesis of Compound a4 (2-Thio-Tetraethylrhodamine-Rhodamine Green) of the Present Invention

Compound a4 of the present invention was synthesized according to the following scheme 7.

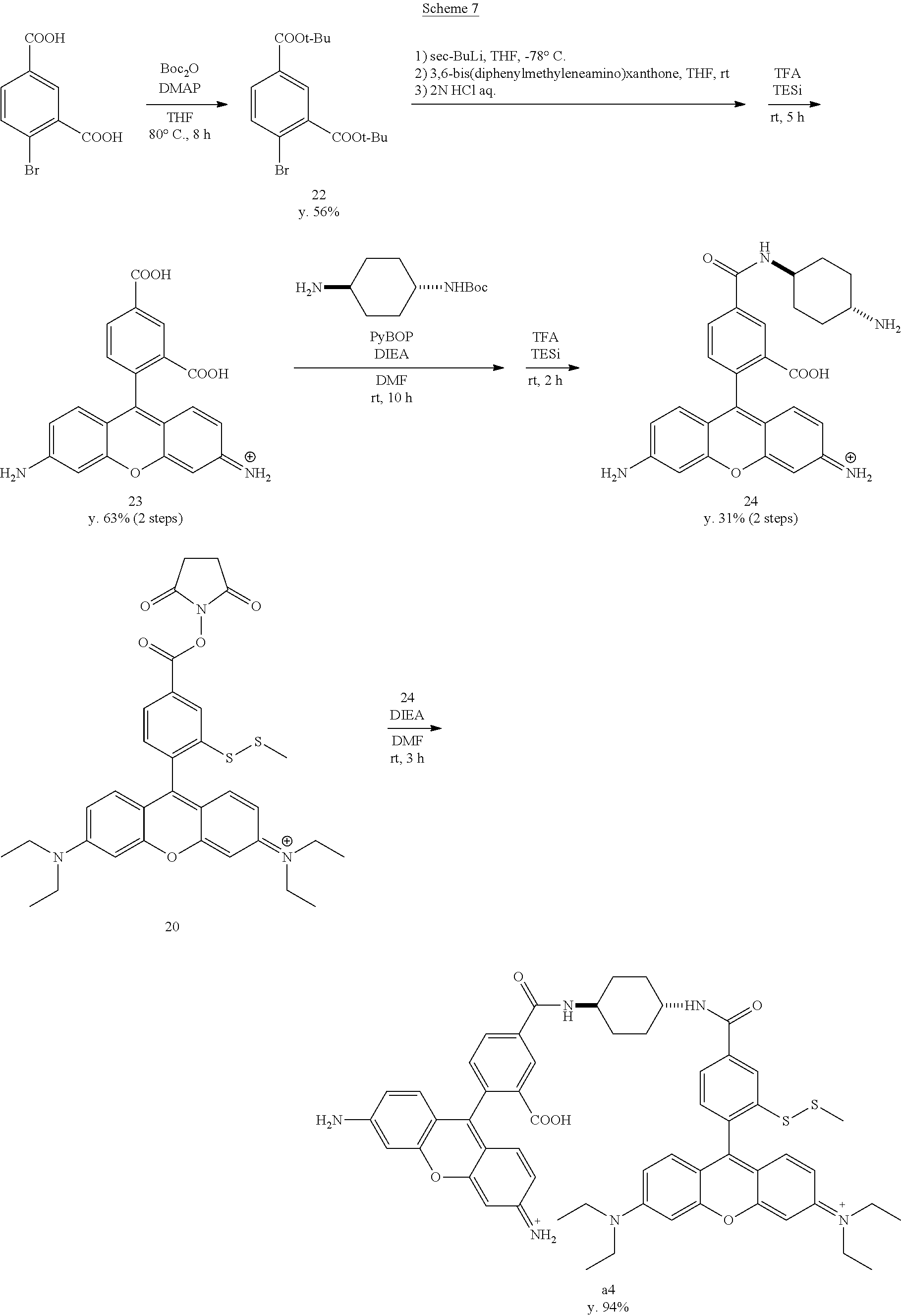

### (1) Synthesis of Compound 22

4-Bromoisophthalic acid (2.45 g, 10 mmol) was dissolved in THF (50 mL), and 4-dimethylaminopyridine (488 mg, 4.0 mmol) and di-tert-butyl dicarbonate (8.72 g, 40 mmol) were added and stirred for eight hours at 80° C. in an argon atmosphere. After distilling off the solvent under reduced pressure, brine was added to the residue. This mixture was extracted by ethyl acetate, and the organic layer was washed with saturated sodium bicarbonate aqueous solution and brine and dried by Na₂SO₄, and the solvent was distilled off under reduced pressure. The residue was purified by column chromatography (silica gel, dichloromethane/n-hexane), and di-tert-butyl-4-bromoisophthalic acid (compound 22, 2.01 g, 5.63 mmol, yield 56%) was obtained.

¹H NMR (300 μMHz, CDCl₃): δ 1.59 (9H, s), 1.61 (9H, s), 7.66 (1H, d, J=8.1 Hz), 7.85 (1H, dd, J=8.1, 2.2 Hz), 8.24 (1H, d, J=2.2 Hz)

¹³C NMR (75 μMHz, CDCl₃): δ 27.86, 28.09, 81.90, 83.01, 125.64, 131.12, 131.55, 132.19, 134.05, 134.43, 164.30, 165.07

### (2) Synthesis of Compound 23

Compound 22 (257 mg, 0.72 mmol) was added to a dried, argon-purged flask and dissolved in dehydrated THF (10 mL). After cooling to −78° C., 1 M sec-butyllithium (540 μL, 0.54 mmol) was added and stirred for two minutes. 3,6-Bis(diphenylmethyleneamino)xanthone (20 mg, 0.036 mmol) dissolved in dehydrated tetrahydrofuran (5 mL) was added slowly at the same temperature, returned to room temperature, and stirred for one hour. The reaction was stopped by 2N HCl aq. This mixture was extracted by dichloromethane, and the organic layer was washed by brine, and the solvent was distilled off under reduced pressure after drying by Na₂SO₄. Trifluoroacetic acid (5.0 mL) and triethylsilane (100 μL) were added to the residue and stirred for five hours at room temperature. After distilling off the solvent under reduced pressure, the residue was purified by HPLC (eluent, from 20% acetonitrile/0.1% trifluoroacetic acid/water (0 min) to 56% acetonitrile/0.1% trifluoroacetic acid/water (15 min); flow rate=5.0 mL/min), and 2,4-dicarboxyrhodamine green (compound 23, 8.5 mg, 0.023 mmol, yield in two steps 63%) was obtained.

¹H NMR (300 μMHz, CD₃OD): δ 6.80-6.81 (4H, m), 7.04 (2H, d, J=9.5 Hz), 7.53 (1H, d, J=8.1 Hz), 8.43 (1H, dd, J=8.1, 1.5 Hz), 8.91 (1H, d, J=1.5 Hz)

HRMS (ESI⁺): calcd for [M]⁺ , 375.0981, found, 375.0958 (−2.3 mmu).

The HPLC chromatogram after purification was as follows. The solution was done with a liner gradient from 20% CH₃CN/0.1% trifluoroacetic acid aq. (0 min) to 80% CH₃CN/0.1% trifluoroacetic acid aq. (15 min) (flow rate=1.0 mL/min).

### (3) Synthesis of Compound 24

Compound 23 (10 mg, 27 μmol) and N-Boc-trans-1,4-cyclohexanediamine (17 mg, 80 μmol) and N,N-diisopropylethylamine (35 mg, 0.027 mmol) and 1H-benzotriazol-1-yl-oxytripyrrolidinophosphonium hexafluorophosphate (PyBOP) (28 mg, 54 μmol) were dissolved in dehydrated DMF (5 mL) and stirred for 10 hours at room temperature. The reaction was then stopped by 1N HCl aq. This mixture was extracted by dichloromethane, and the organic layer was washed with brine, and the solvent was distilled off under reduced pressure after drying by Na₂SO₄. trifluoroacetic acid (2 mL) was added to the residue and stirred for two hours at room temperature. After distilling off the solvent under reduced pressure, the residue was purified by HPLC (eluent, from 20% acetonitrile/0.1% trifluoroacetic acid/water (0 min) to 56% acetonitrile/0.1% trifluoroacetic acid/water (15 min); flow rate=5.0 mL/min), and 2-carboxy-4-aminocyclohexylamide-rhodamine green (compound 24, 4.0 mg, 8.5 μmol, yield in two steps 31%) was obtained.

¹H NMR (300 μMHz, CD₃OD): δ 1.57-1.61 (4H, m), 2.15-2.18 (4H, m), 3.11-3.15 (1H, m), 3.94-3.98 (1H, m), 6.79-6.81 (4H, m), 7.03 (2H, d, J=8.8 Hz), 7.47 (1H, d, J=8.1 Hz), 8.23 (1H, dd, J=8.1, 1.5 Hz), 8.74 (1H, d, J=1.5 Hz)

HRMS (ESI⁺): calcd for [M]+, 471.2032, found, 471.2001 (−3.1 mmu).

The HPLC chromatogram after purification was as follows. The solution was done with a liner gradient from 20% CH₃CN/0.1% trifluoroacetic acid aq. (0 min) to 80% CH₃CN/0.1% trifluoroacetic acid aq. (15 min) (flow rate=1.0 mL/min).

### (4) Synthesis of Compound a4

Compound 20 (2.2 mg, 3.5 μmol) and compound 24 (2.0 mg, 4.2 μmol) and N,N-diisopropylethylamine (4.5 mg, 35 μmol) were dissolved in dehydrated DMF (1.5 mL) and stirred for three hours at room temperature. The reaction was then stopped by 0.1N HCl aq. This mixture was extracted by dichloromethane, and the organic layer was washed with brine, and the solvent was distilled off under reduced pressure after drying by Na₂SO₄. The residue was purified by HPLC (eluent, from 20% acetonitrile/0.1% trifluoroacetic acid/water (0 min) to 80% acetonitrile/0.1% trifluoroacetic acid/water (15 min); flow rate=5.0 mL/min), and compound a4 (3.2 mg, 3.29 μmol, yield 94%) was obtained.

¹H NMR (300 μMHz, CD₃OD): δ 1.32 (12H, t, J=7.0 Hz), 1.66-1.69 (4H, m), 2.16-2.18 (4H, m), 2.39 (3H, s), 3.71 (8H, q, J=7.1 Hz), 4.05 (2H, br s), 6.80-6.82 (4H, m), 7.03-7.10 (6H, m), 7.16 (2H, d, J=9.5 Hz), 7.47 (1H, d, J=7.3 Hz), 7.51 (1H, d, J=8.1 Hz), 7.98 (1H, dd, J=7.3, 1.5 Hz), 8.25 (1H, dd, J=8.1, 2.2 Hz), 8.54 (1H, d, J=1.5 Hz), 8.76 (1H, d, J=2.2 Hz)

HRMS (ESI⁺): calcd for [M]²⁺, 974.3859, found, 974.3873 (+1.4 mmu).

The HPLC chromatogram after purification was as follows. The solution was done with a liner gradient from 20% CH₃CN/0.1% trifluoroacetic acid aq. (0 min) to 80% CH₃CN/0.1% trifluoroacetic acid aq. (15 min) (flow rate=1.0 mL/min).

### Example 2

Na₂S₄ was added to the synthesized compound a1, compound a2, and compound 25, which was obtained by deprotecting compound a4, and the absorption and fluorescence spectra were measured.

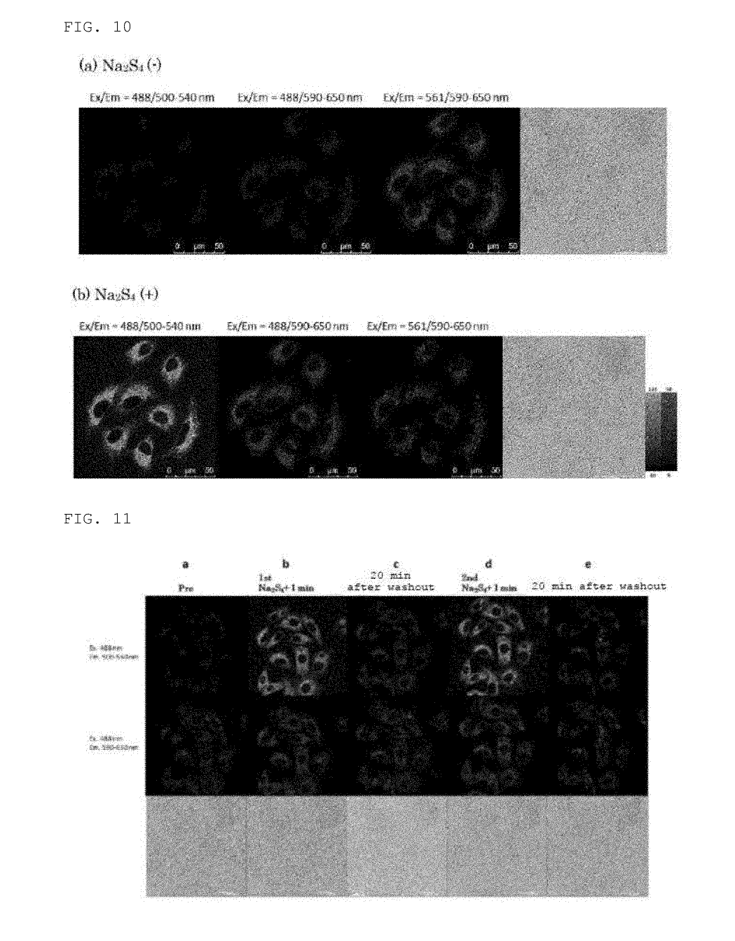

The left drawing of FIG. 7 shows the measurement results of the absorption spectrum of 1 μM of compound a1 in 0.1 M NaPi buffer (pH 7.4) in the presence of 1 mM of GSH before and after addition of 50 μM of Na₂S₄ (containing 0.1% DMSO and 1 mg/mL of BSA as co-solvents). The right drawing of FIG. 7 shows the measurement results of the fluorescence spectrum under the same conditions. The excitation wavelength was 470 nm.

The left drawing of FIG. 8 shows the measurement results of the absorption spectrum of 1 μM of compound a2 in 0.1 M NaPi buffer (pH 7.4) in the presence of 2.5 mM of GSH before and after addition of 50 μM of Na₂S₄ (containing 0.1% DMSO and 1 mg/mL of BSA as co-solvents). The right drawing of FIG. 8 shows the measurement results of the fluorescence spectrum under the same conditions. The excitation wavelength was 470 nm.

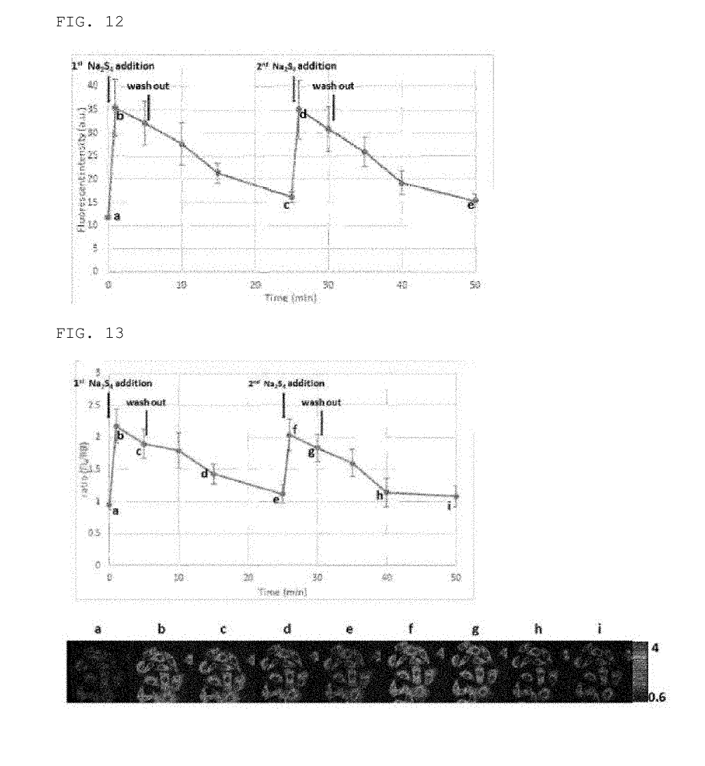

The center drawing of FIG. 9 shows the measurement results of the absorption spectrum of 1 μM of compound 25 in 0.1 M NaPi buffer (pH 7.4) in the presence of 1 mM of GSH before and after addition of 50 μM of Na₂S₄ (containing 0.1% DMSO and 1 mg/mL of BSA as co-solvents). The right drawing of FIG. 9 shows the measurement results of the fluorescence spectrum under the same conditions. The excitation wavelength was 470 nm.

A decrease in absorbance derived from rhodamine and a rise in fluorescence derived from fluorescein of the same magnitude as SSip-1 were seen after Na₂S₄ with compound a1, compound a2, and compound 25, which was obtained by deprotecting compound a4.

### Example 3

Live Cell Imaging by Compound a3

Compound a3 was applied to live cell imaging. The thiol group of compound a3 was protected by disulfide to improve the stability of SSip-1 DA; it is deprotected in the reductive environment within the cells.

FIG. 10 is confocal microscope images of live A549 cells using SSip-1 with 250 μM of Na₂S₄ added. The A549 cells were incubated for three hours by 10 μM of SSip-1 DA (containing 0.03% Purulonic and 0.1% DMSO as a co-solvent). The excitation wavelength was 488 nm. (Laser intensity 20%)/500-540 nm (PMT1000) and 590-650 nm (HyD100)

A rise in fluorescence derived from fluorescein was demonstrated after Na₂S₄ addition. The fluorescence intensity also increased in the rhodamine-derived fluorescence wavelength region, but this was thought to be due to leakage of fluorescein-derived fluorescence. The fluorescence intensity decreased when fluorescence imaging images were acquired at the rhodamine excitation wavelength and fluorescence wavelength (561/590-650 nm).

Reversibility of SSip-1 was Observed in Live Cells.

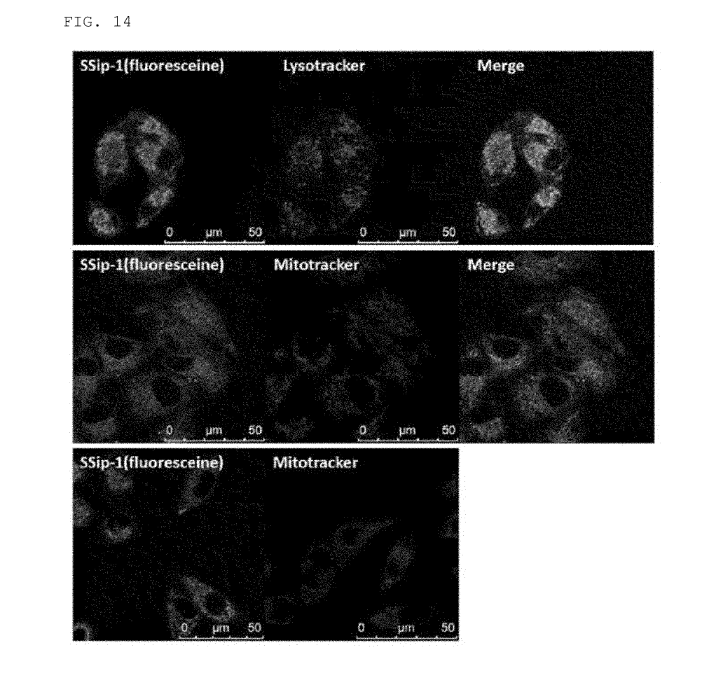

FIG. 11 is confocal microscope images of A549 live cells using SSip-1 with 250 μM of Na₂S₄ added. The confocal microscope images show Na₂S₄ addition followed by washout, then Na₂S₄ again added and followed by washout. The A549 cell incubation conditions and measurement conditions were the same as those of the measurements shown in FIG. 10.

FIG. 12 shows the changes in the fluorescence intensity of A549 live cells incubated by SSip-1 after repeated addition of 250 μM of Na₂S₄.

FIGS. 11 and 12 show that the fluorescence rises within one minute after Na₂S₄ addition, and the fluorescence decreases in about 20 minutes after washout.

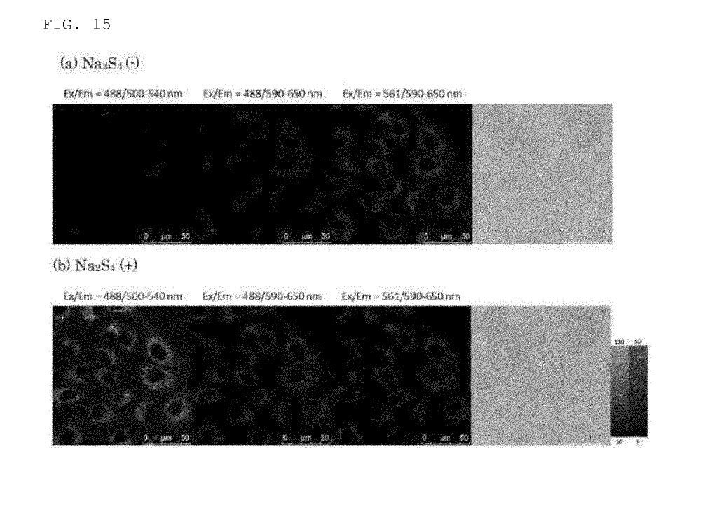

Ratio imaging is also possible with the fluorescence of SSip-1, given that a decrease in rhodamine-derived fluorescence can be observed simultaneously with the rise in fluorescein-derived fluorescence. The upper drawing of FIG. 13 shows the changes in the ratio (FL/RB) of A549 live cells incubated by SSip-1 after repeated addition of 250 μM of Na₂S₄; the upper drawing shows the ratio images.

The localization in cells was studied using compound a3. FIG. 14 shows the results of co-staining of SSip-1 and Lysotracker or Mitotracker. The cells were incubated for one hour by 10 μM of compound a3 and 50 nM of Lysotracker Deep red or 200 nM of Mitotracker Deep red in DMEM (containing 0.03% Purulonic and 0.1% DMSO as a co-solvent). Ex/Em=488 nm (laser intensity 20%)/500-540 nm (PMT1000) and 650 nm/670-700 nm (PMT600)

SSip-1 did not merge with Lysotracker and Mitotracker, but the distribution of SSip-1 was different under the two co-staining conditions. SSip-1 can be said to be localized in the mitochondria in comparison to the localization when only SSip-1 was loaded. SSip-1 can also be kept to the cytoplasm by flushing with Mitotracker, and imaging of sulfane sulfur in the cytoplasm is also thought to be possible.

### Example 4

Live Cell Imaging by Compound a4

Compound a4 was applied to live cell imaging. Compound a4 is obtained by changing the fluorescein site of SSip-1 to rhodamine green, which is thought to improve the photobleaching tolerance.

FIG. 15 is confocal microscope images of A549 live cells using compound a4 with 250 μM of Na₂S₄ added. The A549 cells were incubated for one hour by 10 μM of compound a4 (containing 0.03% Purulonic and 0.1% DMSO as a co-solvent). The excitation wavelength was 488 nm. (Laser intensity 20%)/500-540 nm (PMT1000) and 590-650 nm (HyD100)

A rise in rhodamine green-derived fluorescence intensity and a decrease in rhodamine B-derived fluorescence intensity were demonstrated after Na₂S₄ addition with compound a4.

1. A compound of the following general formula (I): 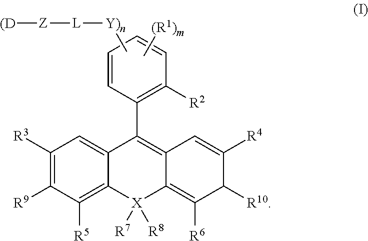
wherein,
R¹ is a hydrogen atom or the same or different monovalent substituents present on the benzene ring;
R² is SH or S—S—R (R is a C₁₋₆ alkyl group);
R³ and R⁴ are, each independently, a hydrogen atom, C₁₋₆ alkyl group, or halogen atom;
R⁵ and R⁶ are, each independently, a hydrogen atom, C₁₋₆ alkyl group, or halogen atom;
R⁷ and R⁸ are, when present, each independently, a C₁₋₆ alkyl group or aryl group;
wherein,
when X is an oxygen atom, R⁷ and R⁸ are not present,
when X is a phosphorus atom, one of —R⁷ and —R⁸ may be ═O;
R⁹ and R¹⁰
(i) are each independently selected from the group consisting of NH₂, monoalkylamino groups, and dialkylamino groups, or
(ii) are each independently selected from the group consisting of a hydroxyl group, and alkoxy group;
X is an oxygen atom, silicon atom, tin atom, germanium atom, or phosphorus atom;
Y is a linking group a with L;
L is a linker;
Z is a linking group b with L;
D is fluorescein, a fluorescein derivative, coumarin, a coumarin derivative, rhodamine, or a rhodamine derivative;
m is an integer of 0-3, n is an integer of 1-4, and m+n=4;
or a salt thereof.

2. The compound according to claim 1, represented by the following general formula (Ia): 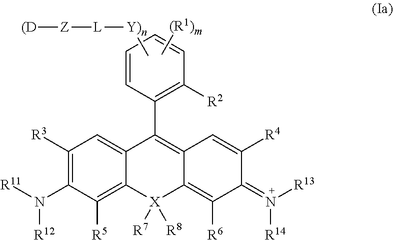
wherein,
R¹-R⁸, X, Y, L, Z, D, m, and n are as defined in general formula (I);
R¹¹ and R¹² are, each independently, a hydrogen atom or C₁₋₆ alkyl group,
R¹¹ or R¹², together with R³ or R⁵, may form a five- to seven-membered heterocycle or heteroaryl containing the nitrogen atoms to which R or R¹² is bonded, may also contain from one to three heteroatoms selected from the group consisting of an oxygen atom, nitrogen atom, and sulfur atom as ring members, and the heterocycle or heteroaryl may also be substituted by a C₁₋₆ alkyl, C₂₋₆ alkenyl, or C₂₋₆ alkynyl, C₆₋₁₀ aralkyl group, or C₆₋₁₀ alkyl-substituted alkenyl group;
R¹³ and R¹⁴ are, each independently, a hydrogen atom or C₁₋₆ alkyl group,
R¹³ or R¹⁴, together with R⁴ or R⁶, may form a five- to seven-membered heterocycle or heteroaryl containing the nitrogen atoms to which R¹³ or R¹⁴ is bonded, may also contain from one to three heteroatoms selected from the group consisting of an oxygen atom, nitrogen atom, and sulfur atom as ring members, and the heterocycle or heteroaryl may also be substituted by a C₁₋₆ alkyl, C₂₋₆ alkenyl, or C₂₋₆ alkynyl, C₆₋₁₀ aralkyl group, or C₆₋₁₀ alkyl-substituted alkenyl group;
or a salt thereof.

3. The compound according to claim 1, represented by the following general formula (Ib): 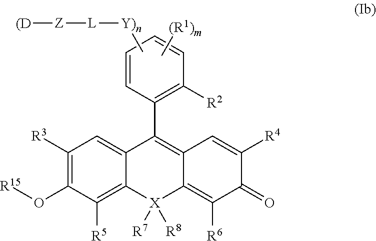
wherein,
R¹-R⁸, X, Y, L, Z, D, m, and n are as defined in general formula (I);
R¹⁵ is a hydrogen atom or C₁₋₆ alkyl group;
or a salt thereof.

4. The compound according to claim 1, wherein the linker is selected from the group consisting of a cycloalkyl group or aryl group, and a salt thereof.

5. The compound according to claim 1, wherein the linking group a is selected from the group consisting of a carbonyl group, alkylcarbonyl group, ester group, alkyl ester group, amino group, alkylamino group, amide group, alkylamide group, isothiocyanate group, sulfonyl chloride group, haloalkyl group, haloacetamide group, azide group, or alkynyl group, and a salt thereof.

6. The compound according to claim 1, wherein the linking group b is selected from the group consisting of a carbonyl group, alkylcarbonyl group, ester group, alkyl ester group, amino group, alkylamino group, amide group, alkylamide group, isothiocyanate group, sulfonyl chloride group, haloalkyl group, haloacetamide group, azide group, or alkynyl group, and a salt thereof.

7. A fluorescent probe comprising the compound according to claim 1 or a salt thereof.

8. A method for detecting sulfane sulfur in cells, the method comprising:
(a) a step for introducing the compound according to claim 1 or a salt thereof into cells; and
(b) a step for measuring the fluorescence emitted by the compound or salt thereof in the cells.
